SEQUENCE LISTING (12 sequences)

SEQ ID NO: 1 - protein, 485 aa, Artificial Sequence
  Chimeric fusion polypeptide of a VHH antigen binding domain (clone L21) and MBP with the di proline linker (Pro-Macrobody 21)
GPSQVQLVESGGGLVQAGGSLRLSCAASGFPVKYEHMYWYRQAPGKEREWVAAINSAGNETHYADSVKGRFTISRDNAKNTVYLQMNSLKPEDTAVYYCNVKDIGWWAAYDYWGQGTQVTVPPLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPG

SEQ ID NO: 2 - protein, 491 aa, Artificial Sequence
  Chimeric fusion polypeptide of a VHH antigen binding domain (clone L51) and MBP with the di proline linker (Pro-Macrobody 51)
GPSQVQLVESGGGSVQAGGSLRLSCAASGSISSITYLGWFRQAPGKEREGVAALATYYGHTYYADSVKGRFTVSLDNAKNTVYLQMNSLKPEDTALYYCAAAYSGIWTPLGVWATYEYWGQGTQVTVPPLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPG

SEQ ID NO: 3 - protein, 126 aa, Vicugna pacos
  E02_12652
QRQLVESGGGSVQSGGSLRLSCAASGFALEDYTVGWFRQAPGKDQEGVSCLSGRDGEVYNAESVMGRFTVSKDSAKSTIYLQMNDLKPEDSAVYFCAADKDVTGRGMCTIPYSDYRGQGTPVTVSS

SEQ ID NO: 4 - protein, 124 aa, Vicugna pacos
  D05_12648
QWQLVESGGGLAQPGGSLRLSCVASGSGMTDIYAMGWYRQAPGKQRELVAWITSGGTTHHANSVKGRFTISRDNAKNTVYLQMNSLKLEDTAVYHCNKVRDFSDFPHWGFMEHWSKGTPVTVSS

SEQ ID NO: 5 - protein, 121 aa, Vicugna pacos
  A11_12648
QWQLVESGGGLVQPGGSLRLSCTFSRGLFNNYPMGWYRQAPGKQRELVAQISAGGASTTYADSVRGRFTISRDNAKKTMYLQMNSLKPEDTAVYLCNTQRYFTSAEARGSWGQGTPVTVSS

SEQ ID NO: 6 - protein, 113 aa, Vicugna pacos
  F10_12652
QLQLVESGGGLVQPGGSLRLSCAASGFTFSDYIMSWVRQAPGKGPEWISSIGNSNDRTMYADSVKGRFTISRDNAKNTLYLQMNSLQFEDTAVYYCGVPAKYWGKGTPVTVSS

SEQ ID NO: 7 - protein, 118 aa, Vicugna pacos
  A09_12649
QLQLVESGGGLVQPGGSLRLSCAASGFTFSAYHMGWYLQAPGKERELVASISSYGSTYYADSVKGRFTVSRDNAKNTVYLQMNSLSPEDTAVYYCNARREGFPPGRYWGKGTPVTVSS

SEQ ID NO: 8 - protein, 120 aa, Vicugna pacos
  H01_12651
QLQLVESGGGLVQPGGSLRLSCAASGSILYFNRMGWYRQAPGKQRELVAAITSGDSTNYADPVKGRFTISRDNAKNTVYLQMNSLKPEDTAVYYCNAKEKGWSFSLYDYWGQGTPVTVSS

SEQ ID NO: 9 - protein, 396 aa, Escherichia coli
  P0AEX9|MALE_ECOLI
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTRITK

SEQ ID NO: 10 - protein, 8 aa, Artificial Sequence
  N-terminus of ABD - peptide linker - C terminus of PPS
VTVPPLVI

SEQ ID NO: 11 - protein, 364 aa, Artificial Sequence
  Di Proline Linker and MBP
PPLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPG

SEQ ID NO: 12 - protein, 367 aa, Artificial Sequence
  N-terminus ABD-PP-MBP
VTVPPLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTPG

(19) United States
(12) Patent Application Publication
(10) Pub. No.: US 2023/0072235 A1
(43) Pub. Date: Mar. 9, 2023

(54) PRO-MACROBODIES FOR THE ENHANCEMENT OF STRUCTURE RESEARCH

(71) Applicant: LEADXPRO AG, Villigen (CH)

(72) Inventors: Stephan SCHENCK, Villigen (CH); Denis BUCHER, Villigen (CH)

(21) Appl. No.: 17/904,346

(22) PCT Filed: Feb. 16, 2021
(86) PCT No.: PCT/EP2021/053794

§ 371 Date: Aug. 16, 2022

(30) Foreign Application Priority Data
Feb. 17, 2020 (EP) 20157617.0

(51) Int. Cl. C07K 19/00; C07K 14/245; G01N 33/68

(52) U.S. Cl. CPC C07K 19/00; C07K 14/245; G01N 33/6803; C07K 2318/00; C07K 2317/569; A61K 39/00

(57) ABSTRACT

The present invention relates to a research tool for structural biology, in particular to enhance the determination of the three-dimensional structure of biological macromolecules. More specifically, the invention serves to improve the overall feasibility of structure determination providing higher resolution and better quality of the three-dimensional structures of proteins by complex-formation with a novel fusion polypeptide.

### BACKGROUND

Structural biology of proteins represents a challenge, in particular for membrane proteins. Structure determination for membrane proteins is therefore lagging behind the structure determination of soluble proteins. Thus, technical advancements are needed, especially for membrane proteins but also for difficult and challenging targets of soluble proteins.

In recent years, advantages in protein production and structure determination technologies have provided important and far-reaching insights into function and pharmacology of membrane proteins. This progress improves structure-based drug design on membrane proteins which are among the major drug targets. Nevertheless, structure determination of membrane proteins, by X-ray crystallography or single particle cryo-electron microscopy (cryo-EM) still represents a significant challenge and technology advancements are needed to improve the quality and cost efficacy as well as reduce the risk of structural information generation. Inherent properties, like conformational flexibility, detergent requirement and the minimal size of the target protein are causative for a demand on technical improvements to increase resolution of membrane protein structures. Cryo-EM is currently a promising method for the determination of the three-dimensional structure of membrane proteins at near-atomic resolution and has evolved into a major technique in structural biology in the past decade. The method is increasingly used for structure-based drug design and holds great promise to become the predominant path for structure determination. Furthermore, proteins that are difficult to crystallize can often be investigated using cryo-EM, in particular membrane proteins. Despite technological advances, one of the most limiting factors for successful structure determination is the size (molecular weight) of the protein of interest, especially for membrane proteins. So far, only few examples of high-resolution structures (below 4 Å resolution) of membrane proteins with a molecular weight of less than 100 kDa have been described [Bai, X. et al. Nature 525, 212-217 (2015); Renaud, J.-P. et al. Nature Reviews Drug Discovery 17, 471-492 (2018)]. Proteins that fall into this size-category are for instance the pharmacologically relevant G-protein coupled receptors (GPCRs) or members of the solute carrier superfamily (SLCs) and more general, monomeric membrane proteins without intrinsic symmetry and only few extra-membrane features (i.e. additional domains on the cytosolic or luminal side). One of the reasons for the difficulties to determine the structure of membrane proteins is the presence of a belt of amorphous detergent (or lipid disc and other specialized polymers) that is needed to keep membrane proteins in solution for single particle imaging. Since the majority of the mass of many membrane proteins is concentrated within this transmembrane area, a large part of the protein is occulted and generally not providing enough features in micrographs for subclass averaging. This factor gets more and more important the smaller the molecular weight of the target protein becomes. Even though the belt of detergents provides contrast to localize particles, the respective orientation of the particle in three-dimensional space is more difficult to assign in the amorphous envelope of detergent. Recent success in obtaining structures of small soluble proteins at resolutions below 3.5 Å, e.g. streptavidin (Fan, X. et al. Nature Communications 10, 2386 (2019)) underline that the detergent belt is a major caveat for structure determination of small membrane proteins.

Detergent belts have also detrimental influence on the crystallization of membrane proteins as they decrease the possibilities for crystal contact formation and require relatively large voids in the crystal lattice to be accommodated in the crystal. These problems have partly been overcome by methods like lipidic cubic phase crystallization (LCP), however vapor diffusion remains an important technique for high resolution structure determination and various methodologies have been deployed to increase the success rate for crystallization and the diffraction properties. Therefore molecular chaperones have also proven very efficient for X-ray crystallography [Zhou, Y., Morais-Cabral, J. H., Kaufman, A. & MacKinnon, R. Nature 414, 43-48 (2001); Dutzler, R., Campbell, E. B. & MacKinnon, R. Science 300, 108 (2003); Brunner, J. D. et al. bioRviv 480863 (2018) doi:10.1101/480863] in vapor diffusion as well as lipidic cubic phase crystallization to provide crystal contacts, and favorable lattice formation.

### PRIOR ART

To overcome the challenges described above, complex formation of the protein of interest with a molecular chaperone can be performed, either to provide additional possibilities for crystal contact formation (in X-ray crystallography) or to enlarge the particle and obtain additional information about the orientation of the particle in space (for cryo-EM). The latter parameter is essential to correctly classify the particle since wrong classification has detrimental effects on the final resolution. To date, Fabs (fragment antigen-binding) of monoclonal antibodies are generally applied for this purpose [Taylor, N. M. I. et al. Nature 546,504-509 (2017); Butterwick, J. A. et al. Nature 560,447-452 (2018)]. They are relatively large (50 kDa) domains of antibodies with a distinct shape, however, also with a variable (clone- and Ig-subclass dependent) intrinsic flexibility between the variable (VHHs of heavy and light chain) and the first constant region. Fabs are generally well visible in EM-micrographs due to their size and distinct donut-like shape, and therefore they fulfill expectations for single particle cryo-EM for several important parameters (size increase of the complex, improved subclass averaging, randomization of particle orientation in space). A major drawback of Fabs for structural purposes is their time-consuming and expensive generation. Further issues are a relatively low number of clones, expensive cell culture methods and hybridoma cultivation for Ig-production. In addition IgGs (and consequently Fabs) have a propensity to bind continuous epitopes, e.g. flexible termini of proteins, however for structural studies binders that recognize three-dimensional non-continuous epitopes are required.

To overcome these drawbacks, molecular chaperones made of single-chain antibodies have been introduced in scientific research [Steyaert, J. & Kobilka, B. K. Current Opinion in Structural Biology 21, 567-572 (2011)]. The unique and compact architecture of single-chain antibody VHH domains (termed nanobodies) results in a number of highly advantageous characteristics compared to normal IgGs. First, camelid or elasmobranch VHHs can easily be cloned from small blood samples because they are built of only one polypeptide chain and not assembled from two gene-products as in classical antibodies, thereby enabling rapid generation of libraries for in-vitro screening and straightforward design of synthetic libraries. Second, this simpler architecture (only one disulfide bond/VHH) enables efficient microbial overexpression/production. And third, the smaller footprint of the paratope of single chain antibodies (three complementarity determining loops (CDRs) compared to six in classical IgGs (2×3 CDRs from heavy and light chain respectively)) predisposes single chain antibodies for binding to cryptic epitopes in clefts and crevices. Importantly, single chain antibodies show similar affinities to their epitopes as classical antibodies. This is mainly due to a relatively extended CDR3 that often provides many interactions with the epitope. The wedge-like structure and the pointed paratope of single chain antibodies as well as their generally small size of only 15 kDa underlie the fact that many single chain antibodies bind to functionally important regions of membrane proteins and selectively recognize a distinct conformation. This reduces motions and conformational heterogeneity in the target protein and often functionally blocks the target protein [Schenck, S. et al. Biochemistry 56, 3962-3971 (2017)]. It is especially this feature that makes single chain antibodies very interesting for structural characterization of proteins and in particular membrane proteins.

Designed ankyrin repeat proteins (DARPins), small proteinaceous binders from synthetic libraries [Binz, H. K. et al. Nature Biotechnology 22, 575-582 (2004)] have been fused to larger proteins (β-lactamase from E. coli) to be used as a crystallization chaperone [Batyuk, A., Wu, Y., Honegger, A., Heberling, M. M. & Plückthun, A. Journal of Molecular Biology 428, 1574-1588 (2016); Wu, Y. et al. Scientific Reports 7, 11217 (2017)]. To the best of our knowledge, for this approach the rigidity and application in cryo-EM have not been evaluated. The fusion polypeptide of a DARPin and scaffold proteins was done by connecting two alpha-helices. For this the N-terminal or C-terminal alpha-helix of a scaffold protein was seamlessly grafted on the terminal helix of a DARPIn. None of the scaffold proteins was connected to a DARPin with beta-sheet architecture at the interface, nor using a proline linker.

Multiple DARPin molecules have been introduced into larger scaffolds with high symmetry to serve as a platform for smaller protein molecules in cryo-EM. These highly complex engineered scaffolds resemble virus particles or large protein complexes and display DARPins (that can be included independent of their target specificity) in up to 12 symmetrically organized copies. These protein complexes are rigid and interconnect with high specificity and affinity, consist of multiple subunits, and require purification and assembly of multiple components [Liu, Y., Gonen, S., Gonen, T. & Yeates, T. O. Proc Natl Acad Sci USA 115, 3362 (2018)]. A similar approach utilized the enzyme aldolase for multimerization of DARPin binding sites [Yao, Q., Weaver, S. J., Mock, J.-Y. & Jensen, G. J. Structure 27, 1148-1155.e3 (2019)]. Here, the connection of the DARPin to aldolase was apparently more flexible than in the modular cage-approach [Liu, Y., Gonen, S., Gonen, T. & Yeates, T. O. Proc Natl Acad Sci USA 115, 3362 (2018)]. In both cases, the binder that is employed is a DARPin, very soluble and well-expressing proteins that have however not been successfully implemented in membrane protein structural biology (e.g. conformation-specific DARPins against GPCRs). Another drawback of these two platforms is the steric limitation that arises from the scaffold itself. Depending on the epitope and the shape of the antigen, clashes with this scaffold are likely to occur. The connection of the DARPIns to the scaffold was made through alpha-helices.

The small size of only 12-15 kDa and compactness of single chain antibodies (more specifically the VHH domains of camelid single chain antibodies) is one of its greatest advantages; however, it represents also a limitation with respect to formation of crystal contacts and especially for the enlargement of target proteins in cryo-EM.

Previously, VHH domains have been enlarged by a loop extension using a fusion protein. This construct was named “megabody” [Laverty, D. et al. Nature 565, 516-520 (2019); Uchański, T. et al. bioRxiv 812230 (2019) doi:10.1101/812230] and is a chimeric protein where the protein hopQ (Uniprot entry B5Z8H1, PDB entry 5LP2), or other scaffold proteins are grafted onto a nanobody in order to increase its size for cryo-EM purposes. Megabodies have been used to structurally characterize heteromeric and homomeric GABA_{A}-receptors [Laverty, D. et al. Nature 565, 516-520 (2019); Masiulis, S. et al. Nature 565, 454-459 (2019)]. Here, the secreted adhesion protein HopQ from Helicobacter pylori was inserted in the first loop after the first beta-strand of the nanobody fold. Importantly, megabodies have thus two connecting peptide strands between the two fused proteins (“internal fusion protein”) and potentially lack the rigidity between the domains [Uchański, T. et al. bioRxiv 812230 (2019) doi:10.1101/812230]. This is indicated in the PDB entries of a α1-β3-γ2-heteromeric GABA receptor in complex with the megabody38 (PDB entries 6HUG, 6HUJ, 6HUK, 6HUO, 6HUP and 6I53) where no model for the grafted hopQ protein was built. We conclude that the grafted protein was insufficiently resolved by cryo-EM and that this chimeric construct provides not enough rigidity to become distinctly visible in micrographs or subclass averages. In the published subclass averages [Laverty, D. et al. Nature 565, 516-520 (2019); Uchański, T. et al. bioRxiv 812230 (2019) doi:10.1101/812230], the density of the hopQ protein is fuzzy, suggesting high flexibility between the nanobody and hopQ moiety. However, the megabody had positive effect on the particle distribution and provided micrographs with many different orientations, which is particularly demanding for Cys-loop receptors like the GABAA receptor.

Recently, enlargement of a camelid VHH was done by a C-terminal fusion to maltose-binding protein (MBP) to provide new crystal contacts for the crystallization of an ion-channel [Brunner, J. D. et al. (2018) doi:10.1101/480863] (PDB entry 6HD8). This chimeric construct was termed “macrobody”. Utilization of this approach has successfully increased the diffractive properties of the crystals and provided a proof for the feasibility to enlarge single chain antibodies in order to design new chaperones. The fusion polypeptide was derived of a recombinant VHH antigen binding domain from an alpaca immunization (also termed nanobody, FIGS. 1A,B [Steyaert, J. & Kobilka, B. K. Current Opinion in Structural Biology 21, 567-572 (2011)]) and a C-terminally linked maltose binding protein (MBP, malE from Escherichia coli, strain K12, Uniprot accession P0AEX9) (FIG. 1C) (Protein Data Bank (PDB) entry 6HD8) (Brunner J. D. et al., BioRxiv 480863, 2018, https://doi.org/10.1101/480863).

The chimeric fusion polypeptide in the entry 6HD8 is shown in FIG. 2 with a Fab fragment of a monoclonal antibody for a size comparison, another commonly used molecular chaperone. The reported fusion polypeptide is connected by a valine linker amino acid between the truncated conserved C-terminal end of the VHH antigen binding domain (FIG. 3A) and an N-terminally truncated MBP starting at amino acid 6 (lysine) by recombinant means. Numbering refers to the processed MBP protein that lacks the signal peptide. This numbering of amino acids excludes the signaling peptide of amino acids 1-26 (the signaling peptide for periplasmic targeting) of the full open reading frame which is removed by the cellular apparatus of E. coli during secretion into the periplasmic space (FIG. 3B). The N-terminal end, in particular the secondary structure of the amino acids 1-64 are shown in FIG. 4. This structural motif is shown for clarity and comprises the most important structural building block to which the VHH antigen binding domain is connected.

The structural conservation of camelid VHH domains is very high, especially at the C-terminus (FIG. 5A). The macrobody was generated as depicted in FIG. 5B and FIG. 6 (Brunner J. D. et al., BioRxiv 480863, 2018, https://doi.org/10.1101/480863). The amino acid valine in the linker is not participating in stable secondary structure elements by hydrogen bonds or hydrophobic interactions (FIG. 7) whereas preceding amino acids in the VHH domain and amino acids in the N-terminus of the MBP moiety are participating in hydrogen bonds of structural beta-sheet elements. The chimeric fusion polypeptide is only connected by a single polypeptide chain between the VHH antigen binding domain and the MBP moiety. The connection is not rigid and can rotate or bend around all three axes (FIG. 7) i.e. the moieties can tumble relative to each other. Unfortunately, the design of the macrobodies has thus an inherent flaw in that the linker of a single polypeptide chain is very flexible and consequently, reduces the chances to form crystal contacts providing good diffraction properties and applicability in cryo-EM due to the lack of visibility in micrographs or subclass averages.

### SUMMARY OF THE INVENTION

Provided is a description of the invention of a chimeric fusion polypeptide comprising a first polypeptide which is an antigen-binding domain and a second polypeptide which is a polypeptide-scaffold connected to the C-terminus of the antigen binding domain, which begins with a beta-sheet architecture comprised of parallel or antiparallel beta-strands and wherein the antigen binding domain and the fused polypeptide-scaffold are linked by a single peptide linker and wherein said peptide linker comprises only proline residues. In particular, the invention comprises a fusion protein with an engineered rigid linker of two proline residues between the C-terminal end of a VHH antigen binding domain and the truncated N-terminus of a second domain that begins with a beta-sheet architecture such as the bacterial periplasmic Maltose Binding Proteins (MBP). This invention is referred as “Pro-Macrobody”. Using computational methods, a fusion protein with an engineered rigid two proline linker following the sequence . . . VTVPPLVI . . . between the C-terminal end of a VHH antigen binding domain and a scaffold protein was designed and evaluated first in silico (italics: antigen binding domain residues, bold: linker, normal letters: enlarging scaffold fusion partner). The antigen binding domain is including all VHH domains of heavy chain antibodies from camelids or elasmobranchs or synthetic libraries derived thereof, or the VHH domain of classical two-chain antibodies (such as IgGs) or synthetic antigen binding domains with immunoglobulin-architecture like monobodies. The scaffold fusion partner polypeptide at the C-terminus of the antigen binding domain is the truncated N-terminus of bacterial periplasmic Maltose Binding Proteins (MBP) in a stricter sense and, in a wider sense all periplasmic binding proteins but in particular the protein malE of Escherichia coli (Uniprot entry P0AEX9). The resulting fusion protein is termed “Pro-Macrobody” and the commonly used acronym would be “PMb” (e.g. monoclonal antibodies are called “MAb” or nanobodies “Nb”). A rigidification and linear connection between the two proteins is achieved by two precisely positioned proline-residues in tandem that are introduced between the two moieties, hence the prefix “Pro-”. The resulting fusion protein, where the VHH antigen binding domain can be replaced with a VHH of any given specificity, serves as a molecular chaperone to enlarge small proteins, in particular membrane proteins. Surprisingly the modification with a di-proline linker results in a major conformational and structural reorganization of the domains relative to each other compared to the structure of the macrobodies lacking the two proline linker. Most remarkably, this new protein is highly rigidified with respect to relative motions between the VHH antigen binding domain and the MBP moiety. The Pro-Macrobodies are compared to macrobodies without the proline-linker and evidence is provided for their much higher rigidity and thus usefulness in structural biology.

Pro-Macrobodies can be used for any structure research, in particular for single particle electron cryo-microscopy (cryo-EM) and X-ray crystallography. This method provides a means to combine the advantageous properties of VHH domains as chaperones for proteins with a fused partner protein module (which is enlarging the VHH) to facilitate cryo-EM or X-ray crystallography structure determination. For cryo-EM the Pro-Macrobody serves to maximize the achievable resolution of the target protein by (i) increasing the molecular weight of the complex resulting in (ii) higher contrast of the particles in micrographs, (iii) better single particle classification due to addition of discernible structural features, (iv) interference with preferred orientations of protein particles on electron microscopy cryo grids and (v) less denaturation of the target protein at air-water interfaces. Therefore, Pro-Macrobodies enable high-resolution structures of proteins without and with bound potential drug compounds, an important method for structure-based drug design. The key advantages of Pro-Macrobodies are the rigid connection that was achieved by linker engineering, the favorable linear extension of the molecule that reduces the chance of clashes with the target protein and finally the universality as any desired antigen binding domain can be integrated into the scaffold as demonstrated by cryo-EM. Further, the antigen-binding properties of the VHH-moiety are preserved (VHH antigen-binding domains and the corresponding Pro-Macrobodies have the same affinities for the respective antigen). Pro-Macrobodies are thus providing an optimized particle recognition feature for alignment and retain the advantageous properties of the VHH-domains (conformation specificity, bacterial production, molecular biology and small footprint paratopes). Described is the design/primary sequence of the chimeric fusion polypeptide, evaluation of possible linkers by all-atom molecular dynamics simulations and evidence for the rigidity of newly engineered Pro-Macrobodies that contain two tandem prolines in the linker using single particle electron microscopy. Additionally a crystal structure of a Pro-Macrobody at a resolution of 2.0 Å is shown that confirmed the predicted fold and provides a high-resolution structure of the designed linker.

Therefore, in a first aspect, the present invention provides a fusion polypeptide comprising a first polypeptide which is an antigen-binding domain and a second polypeptide which is a polypeptide-scaffold, wherein the polypeptide-scaffold comprises parallel or antiparallel beta-strands and the antigen binding domain is linked by a peptide linker at its C-terminus to the N-terminus of the polypeptide-scaffold and wherein said peptide linker consists of one or more proline residues.

In another aspect, the invention relates to an amino acid sequence encoding the fusion polypeptide according to the first aspect of the invention, optionally comprising Sequence ID NO: 001 or Sequence ID NO: 002.

In another aspect, the invention relates to a complex comprising:
i) the fusion polypeptide according to the first aspect of the invention, and
ii) a target protein, wherein said target protein is specifically bound to said fusion polypeptide through the antigen binding domain.

In another aspect, the invention relates to the use of the fusion polypeptide according to the first aspect of the invention, the amino acid sequence, the nucleic acid molecule, the complex of the present invention for structural analyses of a target protein.

In another aspect the use of the fusion polypeptide according to the first aspect of the invention as a medicine.

In another aspect the use of the fusion polypeptide according to the first aspect of the invention for diagnostic purposes.

These and further aspects and preferred embodiments thereof are also additionally defined below in the detailed description and in the claims.

These methods and uses would find widespread use in academic laboratories, pharmaceutical companies, genomics companies, agricultural companies, chemical companies, and in the biotechnology industry.

### DESCRIPTION OF FIGURES

FIG. 1: (A) Secondary structure of a VHH domain with labeling of the beta-strands according to the IMGT global reference nomenclature (Lefranc, M-P., Frontiers in Immunology. 2014, vol 5 (22): 1-22). The complementarity determining regions (CDRs) that bind the epitope are highlighted in black. (B) Crystal structure of a camelid VHH domain (from PDB entry 6HD8). (C) Crystal structure of malE maltose binding protein (MBP) of E. coli (uniprot # P0AEX9; PDB entry 1ANF). In (A)-(C) N- and C-termini are indicated.

FIG. 2: Crystal structure of a chimeric fusion polypeptide of a VHH domain with MBP (a “macrobody”) from the PDB 6HD8 in comparison with the structure of a Fab fragment. Dimensions are indicated in Ångstroms.

FIG. 3: (A) Multiple sequence alignment of the amino acid sequence of six different alpaca VHH domains. CDRs are boxed in grey. Identical residues are indicated in the consensus sequence with an asterisk. The conserved C-terminus of camelid VHH domains is underlined (sequence: VTVSS). The sequence of the VHH that is part of the PDB entry 6HD8 is shown in italics. (B) Amino acid sequence of the maltose binding protein malE of Escherichia coli K12 (Uniprot # P0AEX9). The signaling peptide is shown in the top line and underlined. Lysine 1 of the processed MBP (as occurring in the periplasmic space) and lysine 6 are numbered.

FIG. 4: (A) The conserved structural element of the N-terminal portion of MBP comprising amino acids 1-64. The conserved beta-strands A, B and C as well as the two helices I and II are marked. (B) Topology representation of the N-terminal portion (AA 1-64) of MBP. (C) Enlarged region of the structural element boxed in (B) with indicated hydrogen bonds within the beta strands. (D) Large representation of amino acids 1-64 as shown in (A). This structural building block is conserved throughout the protein family of periplasmic binding proteins and is fused C-terminally to the VHH.

FIG. 5: (A) Superposition of five different camelid VHH domains from PDB entries. While CDRs and the N-terminal portion are not well aligning, the scaffold and C-terminal end are highly conserved on the structural level. The C-terminus is indicated. (B) A VHH and MBP in approximate orientation relative to each other for fusion with each other. The lower panel shows the construct design and amino acids of the linker and the boundaries of MBP used in macrobodies (PDB entry 6HD8).

FIG. 6: Schematic representation of exact boundaries of the macrobody in the PDB entry 6HD8.

FIG. 7: Structure of the linker region of the macrobody in 6HD8. Hydrogen bonds in beta-sheet elements of the VHH and the N-terminal portion of MBP (amino acids 1-64) are shown with distances given in Ångstroms. The linker (Val-Lys) is not involved in hydrogen bonds and can therefore rotate and bend. This is indicated by the arrows.

FIG. 8: Schematic representation of the design for the new chimeric fusion polypeptide of a VHH and MBP using a di-Proline linker at the interface (Pro-Macrobody). Boundaries for fusing the two polypeptides are shown (indicated by scissors) and exact placement for the linker of two proline residues are given.

FIG. 9: (A) Flexibility of designed chimeric fusion polypeptides (macrobodies and Pro-Macrobodies) against LptD in MD simulations. Conformational space explored during a 500 ns MD trajectory initiated from an X-rays structure solved with a double proline, PP-linker (left), and (right) from a simulation model with the linker computationally mutated to a valine and a lysine (VK-linker). Both trajectories were aligned on the VHH structure (residues 1-120). Snapshots are taken every 100 ns and overlaid. A semi-transparent white surface is used to show the extent of the calculated molecular surface explored during the simulations. (B) and (C): Close-up view on the two linkers showing the larger conformational flexibility of the VK-linker by overlaying several snapshots taken from the simulations.

FIG. 10: Essential dynamics analysis of MD trajectories. The correlated motions of alpha carbons were extracted from the trajectories (PP (Pro-Macrobody) structure) after they were aligned on the VHH moiety (residues 1-120). Small arrows describe the global motion resulting from the combination of the first 3 principal components of the covariance matrix. Large arrows indicate the direction where the VHH binds: (A) PP-linker, side-view showing the bending motion; (B) VK-linker for comparison, displaying a much larger bending motion; (C) PP-linker, back-view showing a rotation motion that accompanies the bending; (D) VK-linker showing a large rotation.

FIG. 11: Analysis of macrobody interdomain (VHH to MBP) angle during MD trajectories. (A) Timeline of the interdomain angle in MD initiated from a set of coordinates (x-rays) with the PP-linker formed, and after equilibration (200 ns). The interdomain angle is defined as the angle between the geometrical centers of residue 1-120 (Nb), residue 121-122 (linker), and residue 123-486 (MBP). (B) Distribution of interdomain angles seen in 4 different MD simulations started from two different X-rays structures solved with either a PP-linker (stars), or a VL-linker (empty circles, 6HD8.pdb). In the simulation models, a PP-linker is indicated by a continuous line, while dashed lines correspond to a VK-linker.

FIG. 12: (A) Simulated structure of the di-proline linker (originated from the 6HD8 PDB entry). The amino acids in the linker and one preceding valine residue are shown as ball and sticks. The boxed panel shows a Val-Pro-Pro peptide in comparison where the two consecutive prolines are in trans-configuration. The prolines of the linker in the simulation are present in trans-configuration too. (B) A stretch of a poly-proline II helix for comparison (from the PDB entry 1AWI). All prolines are present in trans-configuration. Note the similar folding to the linker of the simulation. In the poly-proline I helix, that only very rarely occurs in nature, all prolines would be in cis-configuration.

FIG. 13: Original macrobody (from PDBentry 6HD8) in comparison with the Pro-Macrobody (MD-simulation). (A) Macrobody in PDB entry 6HD8 (without antigen). (B) Pro-Macrobody (1 frame from a 500 ns all atom MD simulation). (C) Superposition of (A) and (B) manually aligned on the VHH moiety. The MBP moiety shows a very different conformation. (D) View on the rear as aligned in (C). One helix is framed in green to show the conformational difference between the original macrobody (left) and the modified version with the di-proline linker (right). The MBP moiety is turned approximately 170 degrees counterclock-wise along its long axis when viewed from the C-terminal end.

FIG. 14: Structure statistics of the X-ray structure of Pro-macrobody 21

FIG. 15: (A) X-ray structure of Pro-Macrobody 21 in side view. Linker is shown in ball and sticks. (B) Macrobody (6HD8) with simulated di-proline-linker. The linker is shown as ball and sticks. Note the very similar overall conformation of the X-ray structure and the simulation.

FIG. 16: (A,B) Superposition of the C-alpha trace of the crystallized Pro-Macrobody21 and the simulation with the linker prolines shown in ball and sticks. (C) Enlarged view of the linker, boxed in (B).

FIG. 17: (A) X-ray structure of the Pro-Macrobody 21. (B) Boxed region in (A) shown with the electron density map contoured at 2.5 sigma. The prolines of the linker are indicated. (C) Close-up view of the linker of the Pro-Macrobody 21 with electron density map contoured at 2.5 sigma in two views. The linker prolines are indicated.

FIG. 18: Close up view of the linker interface of the Pro-Macrobody 21 crystal structure. The linker is shown in ball and stick representation. Hydrogen bonds are indicated in dashed lines and corresponding distances in Ångstroms. Note that the hydrogen-bond stabilized regions are only interrupted by the linker that is itself a rigid element.

FIG. 19: Putty representation of temperature factors (B-factors) in the X-ray-structure of Pro-Macrobody 21. High B-factors are indicated by fat cartoon areas whereas low B-factors (low mobility of the atoms) are indicated by thin cartoon drawing. Note that the lowest B-factors in the crystal are observed in the linker region in support of a very rigid connection. Highest B-factors, areas with highest mobility of the atoms, are found at the C-terminal half of MBP and CDRs of the VHH antigen binding domain (in the absence of the antigen).

FIG. 20: Surface representation of the two moieties in the Pro-Macrobody 21. The VHH is shown in light grey and the MBP in dark grey. The linker is shown as spheres. (A) Top view the structure. The linker is not solvent exposed. (B) Bottom view of the structure with visible linker and partly maltose in the binding cleft of MBP. (C) Side view of the Pro-macrobody21. The respective interface areas of the two fused moieties fit very well to each other.

FIG. 21: Binding kinetics of the isolated VHH 21 and Pro-macrobody 21 to the immobilized antigen (NgLptDE) using waveguide interferometry (Creoptix). (A) Binding characteristics of the VHH21. (B) Binding characteristics of the VHH21-PP-MBP (Pro-Macrobody21). (C) Equilibrium binding characteristics of the VHH21. (D) Equilibrium binding characteristics of the VHH21-PP-MBP (Pro-Macrobody21). Note that the kinetic parameters are almost unchanged. The off-rate (kd) is slightly larger for the Pro-Macrobody.

FIG. 22: Binding kinetics of the isolated VHH 51 and Pro-Macrobody 51 to the immobilized antigen (NgLptDE) using waveguide interferometry (Creoptix). (A) Binding characteristics of the VHH51. (B) Binding characteristics of the VHH51-PP-MBP (Pro-macrobody21). (C) Equilibrium binding characteristics of the VHH51. (D) Equilibrium binding characteristics of the VHH51-PP-MBP (Pro-Macrobody51). Note that the kinetic parameters are almost unchanged.

FIG. 23: Size exclusion chromatogram of a Pro-Macrobody 21-NgLptDE complex (dashed line) in comparison with NgLptDE (solid line) on a Superdex 200 5/150 column. The complex of NgLptDE and Promacrobody21 elutes earlier from the column, indicative for a higher molecular weight. The shift in elution volume is shown in the lower panel for clarity. The filled circle and asterisk denote uncomplexed NgLptDE and the Pro-Macrobody21/NgLptDE complex respectively. The arrow indicates excess Pro-Macrobody21 eluting much later from the column.

FIG. 24: Preparation of a ternary complex of Pro-Macrobodies 21 and 51 with NgLptDE for structure determination using cryo-EM. (A) Size exclusion chromatogram of the ternary complex injected on a Superdex 200 10/300 column. The fraction used for cryo-EM is highlighted with a light grey bar. Excess Pro-Macrobodies are well separated from the complex. (B) SDS-PAGE analysis of the eluted fractions from (A). Unbound Pro-Macrobodies (added to NgLptDE in 3-fold molar excess) are well separated from the ternary complex. Pro-Macrobodies 21 and 51 have near identical molecular weights and cannot be resolved on SDS-PAGE. Residual free MBP (contaminant) is also separated. Additional contaminants visible in the gel are present at ˜40 and ˜30 kDa, but did not negatively affect structure determination.

FIG. 25: Cryo-EM subclass averages (2D projections) of NgLptDE-Pro-Macrobodies21/51 ternary complex. The complex can be seen in different orientations and bound Pro-Macrobodies are clearly visible (arrows).

FIG. 26: Close-up view of two subclass averages from FIG. 25. Subdomains within the Pro-Macrobodies (VHH-PP-MBP) can be clearly distinguished. The asterisk marks the VHH antigen-binding domain, the arrow marks the N-terminal half of MBP, and the arrowhead the C-terminal half of MBP. Maltose is bound in a cleft between the N- and C-terminal halves of MBP. A surface model downscaled to ˜8 Angstroms from the crystal structure of the Pro-macrobody L21 is shown on the right (maltose-bound form). The subdomains are indicated with the same symbols as in the left panel.

FIG. 27: Three-dimensional Cryo-EM map of the ternary complex of NgLptDE/Pro-Macrobody 21/Pro-Macrobody 51 with subdomains of the Pro-Macrobodies labeled. NgLptDE is shown in light grey.

FIG. 28: Comparison of the structures of the simulated Pro-Macrobody, the X-ray structure of Pro-Macrobody 21 and the EM-structures of Pro-Macrobodies 21 and 51. (A) A single frame of the simulated Pro-Macrobody (from 6HD8 structure). (B) X-ray structure of Pro-Macrobody 21 at 2 Å resolution (left) and simulated lower resolutions (right panels). (C) Pro-Macrobody 21 Cryo-EM structure. The right panel is a view rotated by 180° degrees. (D) Pro-Macrobody 51 Cryo-EM structure. The right panel is a view rotated by 180° degrees. All structures are shown in the same orientation, except for the right panels in (C) and (D). Note the high similarity of the conformations. The EM-structures are in the absence of bound maltose, explaining the open conformation of the MBP (see also FIG. 29). Pro-Macrobodies 21 and 51 have a very similar shape, providing evidence for the universal character of the Pro-Macrobody design.

FIG. 29: X-ray structure of Pro-Macrobody 21 fitted into the EM map of the ternary complex of NgLptDE/Pro-Macrobody 21/Pro-macrobody 51. The X-ray-structure is the maltose-bound form and therefore more closed whereas the cryo-EM structure is the apo-form and the N- and C-terminal lobes of MBP are forming a larger gap between each other. Pro-Macrobody 51 is not shown, but the very similar conformation of Pro-Macrobody 51 is evident from comparison in FIG. 28.

FIG. 30: Global resolution of NgLptDE and the NgLptDE/Pro-Macrobody ternary complex from cryo-EM. The gold standard fourier shell correlation is shown (GSFSC) with a FSC threshold of 0.143. Using Pro-Macrobodies, the resolution of the map could be improved by 1.2 Å compared to a structure obtained with a comparable number of particles. Importantly the resolution is improved on a level that is crucial for visualization of side chains.

FIG. 31: Comparison of cryo-EM maps for NgLptDE that were obtained with uncomplexed NgLptDE (1^{st} dataset) and from a NgLptDE/Pro-Macrobody 21/Pro-Macrobody 51 ternary complex (2^{nd} dataset). The 2^{nd} dataset show a much higher resolution, in central areas of the transmembrane area below 3 Å, whereas the uncomplexed NgLptDE has a maximal resolution of 4-4.5 Å. For both maps similar numbers of particles were used.

FIG. 32: (A) Subclass averages of a complex of NgLptDE and macrobodies (same VHH domains as in FIG. 31 (VHH21/51)) but with a VK linker instead of a PP linker as in Pro-Macrobodies. The MBP moiety is indicated by arrows. (B) In the lower panel a three-dimensional reconstruction from this dataset is shown, with an overall resolution that is not beyond the dataset of uncomplexed NgLptDE and compared to the dataset with Pro-Macrobodies (C). Pro-Macrobodies are indicated by arrowheads.

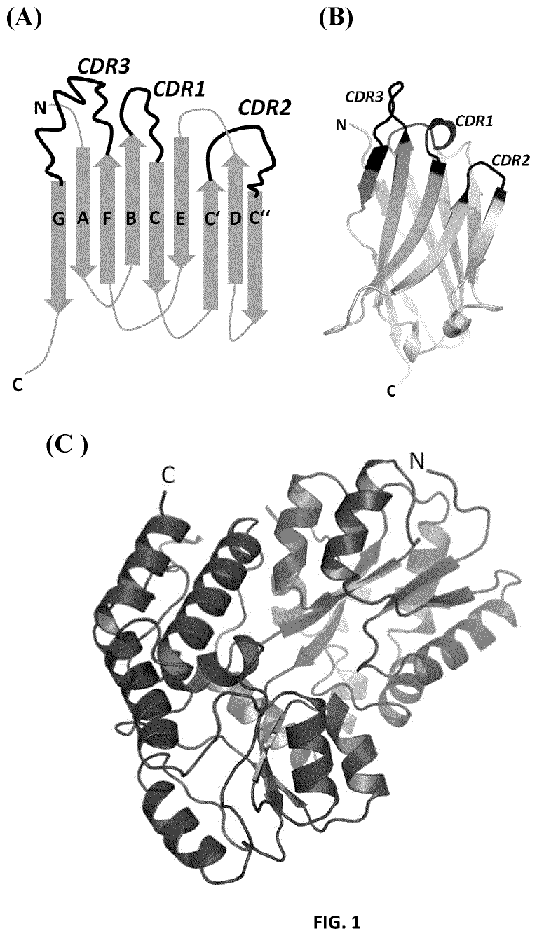

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and very preferred embodiments should apply to all aspects and other embodiments, preferred embodiments and very preferred embodiments irrespective of whether it is specifically again referred to or its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise.

In an aspect, the present invention provides a fusion polypeptide comprising a first polypeptide which is an antigen-binding domain and a second polypeptide which is a polypeptide-scaffold, wherein the polypeptide-scaffold comprises parallel or antiparallel beta-strands and the antigen binding domain is linked by a peptide linker at its C-terminus to the N-terminus of the polypeptide-scaffold and wherein said peptide linker consists of one or more proline residues.

As used herein, the “antigen-binding domain” is limited only by binding to the target antigen. As the antigen binding domain, domains of any structure can be used as long as they bind to the target antigen. One suitable example of the antigen binding domain of the present invention is a single domain antibody (sdAb).

The term “polypeptide” refers to any sequence of two or more amino acids, regardless of length, post-translation modification, or function. Polypeptides can include natural amino acids and non-natural amino acids. In one embodiment the fusion protein comprises adjacent strands of the beta sheet structure and may be parallel or anti-parallel to each other.

As used herein, the term “linker” refers to a linkage between two elements, e.g., protein domains. A linker can be a covalent bond or a spacer. The term “bond” refers to a chemical bond, e.g., an amide bond or a disulfide bond, or any kind of bond created from a chemical reaction, e.g., chemical conjugation. The term “spacer” refers to a moiety (e.g., a polyethylene glycol (PEG) polymer) or an amino acid sequence (e.g., a 3-200 amino acid, 3-150 amino acid, or 3-100 amino acid sequence) occurring between two polypeptides or polypeptide domains to provide space and/or flexibility between the two polypeptides or polypeptide domains.

The “C terminus” or “carboxy terminus” are used herein interchangeably and are defined herein as they are normally used in the art. An amino acid structure contains a carbon atom known as the carbon to which is bonded an amine group, a carboxylic acid group and a side chain. The N-terminus (also known as the amino-terminus, NH₂-terminus, N-terminal end or amine-terminus) is the start of a protein or polypeptide referring to the free amine group (−NH₂) located at the end of a polypeptide.

In one embodiment the present invention provides the fusion polypeptide wherein the polypeptide-scaffold is a maltose binding protein.

In another embodiment the present invention provides the fusion polypeptide wherein the polypeptide-scaffold is Escherichia coli maltose binding protein, Uniprot entry P0AEX9.

In another embodiment the present invention provides the fusion polypeptide wherein the peptide linker consists of one, two, three or four proline residues.

In another embodiment the present invention provides the fusion polypeptide wherein the peptide linker consists of two proline residues. As indicated, such a rigid linker of two proline residues between the C-terminal end of an antigen binding domain, preferably a VHH antigen binding domain, and the polypeptide-scaffold, preferably a second domain that begins with a beta-sheet architecture such as the truncated N-terminus of the bacterial periplasmic Maltose Binding Proteins (MBP).

In another embodiment, the present invention provides the fusion polypeptide, wherein the C-terminal end of said antigen binding domain consists of the amino acid sequence VTV. Such C-terminus sequence is typically present in all VHH domains of heavy chain antibodies from camelids or elasmobranchs or synthetic libraries derived thereof, or the VHH domain of classical two-chain antibodies (such as IgGs) or synthetic antigen binding domains with immunoglobulin-architecture like monobodies. In another embodiment, the present invention provides the fusion polypeptide wherein, the C-terminal end of said antigen binding domain consists of the amino acid sequence VTV, wherein said antigen binding domain is a VHH antigen binding domain.

In another embodiment, the present invention provides the fusion polypeptide wherein, the N-terminal end of said polypeptide-scaffold consists of the amino acid sequence LVI. Preferably, said polypeptide-scaffold, said amino acid sequence LVI is comprised by and typically begins a beta-sheet architecture such as the truncated N-terminus of the bacterial periplasmic Maltose Binding Proteins (MBP). The scaffold fusion partner polypeptide at the C-terminus of the antigen binding domain is the truncated N-terminus of bacterial periplasmic Maltose Binding Proteins (MBP) in a stricter sense and, in a wider sense all periplasmic binding proteins but in particular the protein malE of Escherichia coli (Uniprot entry P0AEX9). In another embodiment, the present invention provides the fusion polypeptide wherein, the N-terminal end of said polypeptide-scaffold consists of the sequence LVI, wherein said polypeptide-scaffold is a Escherichia coli maltose binding protein, Uniprot entry P0AEX9, and wherein preferably said polypeptide-scaffold is a truncated N-terminus of the bacterial periplasmic Maltose Binding Proteins (MBP).

In another embodiment, the present invention provides the fusion polypeptide wherein the C-terminal end of said antigen binding domain is linked by said peptide linker, which consists of two prolines to the N-terminus of the polypeptide-scaffold, and wherein the C-terminal end of said antigen binding domain linked by said peptide linker to the to the N-terminus of the polypeptide-scaffold comprises the amino acid sequence VTVPPLVI (SEQ ID NO: 10).

In another embodiment, the present invention provides the fusion polypeptide comprises the amino acid sequence VTVPPLVI (SEQ ID NO: 10).

In another embodiment, the present invention provides the fusion polypeptide, wherein the C-terminal end of said antigen binding domain, wherein said antigen binding domain is a VHH antigen binding domain, is linked by said peptide linker, which consists of two prolines, i.e. wherein said peptide linker is di-amino acid PP-linker, to the N-terminus of the polypeptide-scaffold, wherein said polypeptide-scaffold is a Escherichia coli maltose binding protein, Uniprot entry P0AEX9, and preferably wherein said polypeptide-scaffold is a truncated N-terminus of the bacterial periplasmic Maltose Binding Proteins (MBP), wherein the C-terminal end of said antigen binding domain linked by said peptide linker to the to the N-terminus of the polypeptide-scaffold comprises the amino acid sequence VTVPPLVI (SEQ ID NO: 10). A rigidification and linear connection between the two proteins is achieved by two precisely positioned proline-residues. The resulting fusion protein, where the VHH antigen binding domain can be replaced with a VHH of any given specificity, serves as a molecular chaperone to enlarge small proteins, in particular membrane proteins. Surprisingly the modification with a di-proline linker results in a major conformational and structural reorganization of the domains relative to each other compared to the structure of the macrobodies lacking the two proline linker. Most remarkably, this new preferred fusion protein linkage is highly rigidified with respect to relative motions between the VHH antigen binding domain and the polypeptide-scaffold, preferably the MBP moiety.

In another embodiment, the present invention provides a fusion polypeptide, wherein the peptide linker connects the C-terminal portion of the antigen-binding domain with the N-terminal portion of the polypeptide-scaffold.

In another embodiment, the present invention provides, a fusion polypeptide, wherein the peptide linker connects the conserved and truncated carboxy-terminus of single chain antibody VHH domains of camelids after the conserved sequence Valine-Threonine-Valine (VTV) at the end of the beta-strand G to the truncated amino-terminus of the Escherichia coli maltose binding protein starting from the amino acid position leucine 7, the first amino acid in the beta-strand A of maltose binding protein.

As used herein, the term “amino acid positions” refers to the position numbers of amino acids in a protein or protein domain.

In another embodiment, the present invention provides a fusion polypeptide, wherein the C-terminus of the VHH of the antigen binding domain comprises three amino acids Valine-Threonine-Valine and the N-terminus of the maltose binding protein comprises three amino acids Leucine-Valine-Isoleucine.

In another embodiment, the present invention provides a fusion polypeptide, wherein the carboxy terminus of the peptide linker is fused to the first amino acid of the most amino-terminal beta-strand of the polypeptide-scaffold.

In another embodiment the present invention provides a fusion polypeptide, wherein the polypeptide-scaffold comprises a polypeptide of the superfamily of periplasmic binding proteins of Interpro entry IPR025997, or a polypeptide of the superfamily of periplasmic binding protein-like I integral of Interpro entry IPR028082, or a periplasmic binding protein domain Pfam domain Peripla_BP_4 of PF13407.

In another embodiment the present invention provides a fusion polypeptide of Interpro superfamilies IPR025997 and IPR028082 or Pfam domain PF13407 wherein there is at least one amino acid substitution of the original database entries occurring in this scaffold.

In another embodiment the present invention provides a fusion polypeptide comprising additional fusion polypeptides that are integrated, either internally at any position or fused to the carboxy-terminal end of the polypeptide-scaffold.

In another embodiment the present invention provides a fusion polypeptide wherein the fused polypeptide belonging to the Interpro superfamilies IPR025997 and IPR028082 or containing a Pfam domain PF13407, are truncated before its native carboxy-terminal end.

In another embodiment, the present invention provides a fusion polypeptide, wherein said peptide linker and said polypeptide-scaffold comprises, preferably consist of, the amino acid sequence SEQ ID NO:11.

In another embodiment, the present invention provides a fusion polypeptide, wherein said fusion polypeptide comprises the amino acid sequence SEQ ID NO:11.

(SEQ ID NO: 11)
PPLVIWINGDKGYNGLAEVGKKFEKDTGIKVTVEHPDKLEEKFPQVAATGD
GPDIIFWAHDRFGGYAQSGLLAEITPDKAFQDKLYPFTWDAVRYNGKLIAY
PIAVEALSLIYNKDLLPNPPKTWEEIPALDKELKAKGKSALMFNLQEPYFT
WPLIAADGGYAFKYENGKYDIKDVGVDNAGAKAGLTFLVDLIKNKHMNADT
DYSIAEAAFNKGETAMTINGPWAWSNIDTSKVNYGVTVLPTFKGQPSKPFV
GVLSAGINAASPNKELAKEFLENYLLTDEGLEAVNKDKPLGAVALKSYEEE
LAKDPRIAATMENAQKGEIMPNIPQMSAFWYAVRTAVINAASGRQTVDEAL
KDAQTPG.

In another embodiment, the present invention provides a fusion polypeptide, wherein said antigen-binding domain, peptide linker and said polypeptide-scaffold comprises the amino acid sequence SEQ ID NO:12:

In another embodiment, the present invention provides a fusion polypeptide, wherein said fusion polypeptide comprises the amino acid sequence SEQ ID NO:12.

(SEQ ID NO: 11)
VTVPPLVIWINGDKGYNGLAEVGKKFEKDTGIKVTVEHPDKLEEKFPQVAA
TGDGPDIIFWAHDRFGGYAQSGLLAEITPDKAFQDKLYPFTWDAVRYNGKL
IAYPIAVEALSLIYNKDLLPNPPKTWEEIPALDKELKAKGKSALMFNLQEP
YFTWPLIAADGGYAFKYENGKYDIKDVGVDNAGAKAGLTFLVDLIKNKHMN
ADTDYSIAEAAFNKGETAMTINGPWAWSNIDTSKVNYGVTVLPTFKGQPSK
PFVGVLSAGINAASPNKELAKEFLENYLLTDEGLEAVNKDKPLGAVALKSY
EEELAKDPRIAATMENAQKGEIMPNIPQMSAFWYAVRTAVINAASGRQTVD
EALKDAQTPG.

In another embodiment the present invention provides the fusion polypeptide, wherein the antigen binding domain comprises an immunoglobulin-like fold.

The immunoglobulin (Ig)-like domain is a protein domain that is similar in amino acid sequence and structure to the Ig domains of immunoglobulins (Edelman, 1987; Williams & Barclay, 1988). Structurally, Ig domains possess a distinctive immunoglobulin fold composed of 70-110 amino acids.

In another embodiment the present invention provides the fusion polypeptide, wherein the antigen-binding domain comprises an immunoglobulin (Ig) domain.

Ig domains are 70-110 amino acids in length with a distinct overall structure referred to as the Ig fold. This fold consists of two β sheets each made up of short antiparallel β strands. In the majority of Ig domains, the two β sheets are joined by a disulphide linkage.

In another embodiment the present invention provides the fusion polypeptide, wherein the antigen-binding domain is a VHH antigen-binding domain.

In another embodiment the present invention provides the fusion polypeptide, wherein the antigen-binding domain comprises a camelid VHH or elasmobranch VHH, or shark VHH, or ray VHH or skate VHH or sawfish VHH or VHH domains from heavy or light chain of mammalian antibodies or monobodies.

In another embodiment the present invention provides a fusion polypeptide, wherein the VHH domain originates from a single-chain antibody that was isolated from a member of the family of camelidae (i.e. Llama spp., Vicugna spp., or Camelus spp.).

In another embodiment the present invention provides a fusion polypeptide, wherein the VHH-antigen binding domain is a VHH domain of a heavy chain or light chain of a classical vertebrate Ig-antibody.

In another embodiment the present invention provides a fusion polypeptide, wherein the VHH-antigen binding domain is replaced with a monobody (synthetic binders built on a fibronectin type III domain) or is an immunoglobulin (Ig).

In another embodiment the present invention provides a fusion polypeptide, wherein the VHH-antigen binding domain is selected from a synthetic library that was generated by recombinant technology, or PCR or presented in recombinant Saccharomyces cerevisiae or that is presented in recombinant phages.

In another aspect the invention provides an amino acid sequence encoding the fusion polypeptide of the present invention. In one embodiment the amino acid sequence comprises Sequence ID NO: 001 or Sequence ID NO: 002.

The amino acid sequence ID NO: 001:

GPSQVQLVESGGGLVQAGGSLRLSCAASGFPVKYEHMYWYRQAPGKEREWV
AAINSAGNETHYADSVKGRFTISRDNAKNTVYLQMNSLKPEDTAVYYCNVK
DIGWWAAYDYWGQGTQVTVPPLVIWINGDKGYNGLAEVGKKFEKDTGIKVT
VEHPDKLEEKFPQVAATGDGPDIIFWAHDRFGGYAQSGLLAEITPDKAFQD
KLYPFTWDAVRYNGKLIAYPIAVEALSLIYNKDLLPNPPKTWEEIPALDKE
LKAKGKSALMFNLQEPYFTWPLIAADGGYAFKYENGKYDIKDVGVDNAGAK
AGLTFLVDLIKNKHMNADTDYSIAEAAFNKGETAMTINGPWAWSNIDTSKV
NYGVTVLPTFKGQPSKPFVGVLSAGINAASPNKELAKEFLENYLLTDEGLE
AVNKDKPLGAVALKSYEEELAKDPRIAATMENAQKGEIMPNIPQMSAFWYA
VRTAVINAASGRQTVDEALKDAQTPG

The amino acid sequence ID NO: 002:

GPSQVQLVESGGGSVQAGGSLRLSCAASGSISSITYLGWFRQAPGKEREGV
AALATYYGHTYYADSVKGRFTVSLDNAKNTVYLQMNSLKPEDTALYYCAAA
YSGIWTPLGVWATYEYWGQGTQVTVPPLVIWINGDKGYNGLAEVGKKFEKD
TGIKVTVEHPDKLEEKFPQVAATGDGPDIIFWAHDRFGGYAQSGLLAEITP
DKAFQDKLYPFTWDAVRYNGKLIAYPIAVEALSLIYNKDLLPNPPKTWEEI
PALDKELKAKGKSALMFNLQEPYFTWPLIAADGGYAFKYENGKYDIKDVGV
DNAGAKAGLTFLVDLIKNKHMNADTDYSIAEAAFNKGETAMTINGPWAWSN
IDTSKVNYGVTVLPTFKGQPSKPFVGVLSAGINAASPNKELAKEFLENYLL
TDEGLEAVNKDKPLGAVALKSYEEELAKDPRIAATMENAQKGEIMPNIPQM
SAFWYAVRTAVINAASGRQTVDEALKDAQTPG

In a further aspect the invention provides a chemically modified, covalently labeled fusion polypeptide of the present invention.

In another aspect the present invention provides a complex comprising:
i) the fusion polypeptide of the present invention, and
ii) a target protein, wherein said target protein is specifically bound to said fusion polypeptide.

In one embodiment the complex of the present invention, wherein said target protein is bound to the antigen binding domain of said fusion polypeptide.

In another embodiment the complex of the present invention, wherein the target protein has a molecular weight of less than 100 kDa. In another embodiment the complex of the present invention, wherein the target protein has a molecular weight of less than 90 kDa, or 80 kDa, or 70 kDa or 60 kDa or 50 kDa or 40 kDa or 30 kDa, or 10 kDa.

In another aspect the invention provides the use of the fusion polypeptide, the amino acid sequence and the complex of the present invention for structural analyses of a target protein.

In one embodiment said structural analysis comprises single particle cryo-electron microscopy (cryo-EM) or X-ray crystallography. In another embodiment said structural analysis comprises single particle cryo-EM or X-ray crystallography or negative staining TEM or electron diffraction or NMR or any other structure determination technology.

In another aspect the invention provides the use of the fusion polypeptide according to the present invention as a medicine.

In another aspect the invention provides the use of the fusion polypeptide according to the present invention for diagnostic purposes.

In another aspect the present invention provides a fusion polypeptide of the present invention comprising Interpro entries
i) Periplasmic binding protein/LacI sugar binding domain (IPR001761)
ii) Receptor, ligand binding region (IPR001828)
iii) Leucine-binding protein domain (IPR028081)
iv) Arabinose metabolism transcriptional repressor, ligand-binding domain

### EXAMPLE I

### Evaluation of a Chimeric Fusion Polypeptide (Macrobody) and Design of New Chimeric Fusion Polypeptides (Pro-Macrobody) for Increased Rigidity by Computer Simulations

Described is the workflow for analysis of motions in chimeric fusion polypeptides using computer simulations (all-atom molecular dynamics simulations) and the simulation of in-silico amino acid exchanges (mutants) in the linker-region. The chimeric fusion polypeptides with amino exchanges in the linker region were subjected to all atom-molecular dynamics simulations (MD simulations) and their motions were quantified. All MD simulations are fully atomistic (ie. Each atom is simulated and not groups of atoms/molecules) and were conducted with Desmond and the OPLS3 force field with explicit solvent at room temperature (300K, or 26.85° C.). For all simulations a prior equilibration of 300 ns was conducted prior to 500 ns of production.

Simulation Models

Four different VHH-MBP fusion polypeptide simulation models were built starting from two different sets of coordinates. The first set of coordinates was built from the PDB data base code 6HD8.pdb structure (macrobody, here VK structure), which was simulated with a Val121 and Lys122 linker (from now on VK-linker, numbering refers to the Protein Data Bank (PDB) entry 6HD8). The second set of coordinates was built from a new structure simulated (description below) of a VHH-MBP fusion polypeptide where the antigen is the protein LptD of Neisseria gonnorhoeae (Uniprot accession A0A1D3INQ1) (Pro-macrobody, here PP structure) and a Pro122 Pro123 linker (from now on PP-linker). The VK structure was simulated either in its original state or after changing its linker to a PP-linker. Similarly, the PP structure was simulated either in its original state or after changing its linker to the VK-linker.

The exact design of the chimeric VHH-MBP fusion polypeptide comprising a di-Proline linker with all important boundaries is depicted in FIG. 8. This design was used for in-silico studies and for overexpression in E. coli and the crystallization.

Results

MD simulations initiated from both VK and PP structures and with both VK and PP linkers showed the great impact of the linker residue type on the dynamics of the VHH-MBP fusion polypeptides. Initial results were obtained for the VK structure and suggested that the PP-linker could lead to a significantly more rigid chaperone. Subsequently, a VHH-MBP fusion polypeptide was generated in silico with a PP-linker to test this hypothesis and a new VHH-MBP fusion polypeptide was produced in E. coli where the VHH antigen binding domain is binding the said LptD protein. The X-rays structure for this designed Pro-macrobody was solved (Section III) and shown to be in agreement with the predicted structure from MD simulations (RMSD<2 Å). This set of coordinates (PP structure) was then seeded to run additional MD simulations, including for comparison a simulation model where the VK-linker was re-introduced. Clear agreement was obtained in both cases, showing that the linker is able to modify the dynamics irrespectively of the initial set of coordinates chosen to run the simulations.

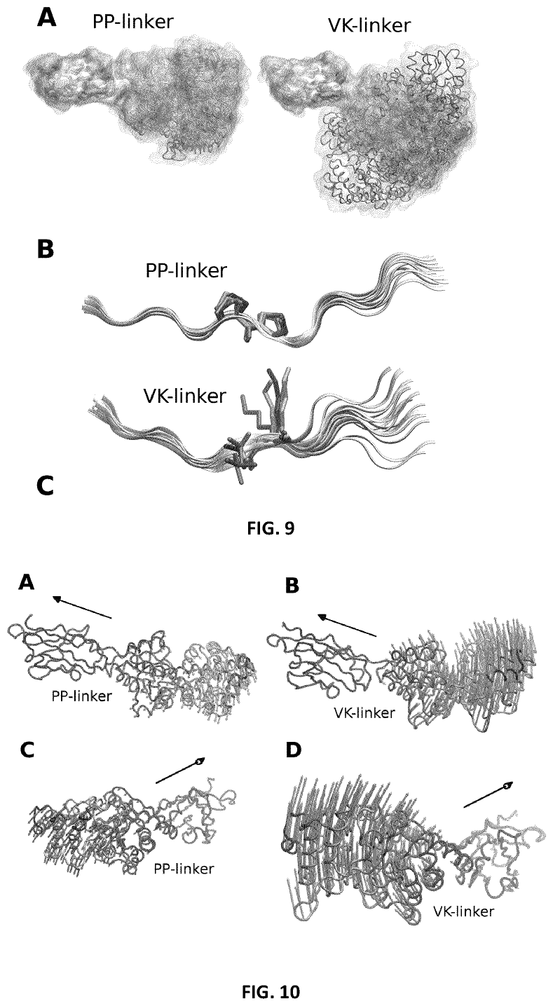

In FIG. 9A, the rigidity of both VK- and the designed PP-linker is shown using models built from the VK structure. Much larger bending is observed around the linker, which can be observed also by zooming in on the linker (FIG. 9B).

In FIG. 10, both a bending and rotational motions are shown to occur between the VHH and MBP, with much reduced flexibility in the case of the PP linker. The visualization is deduced from a normal mode analysis of the simulations.

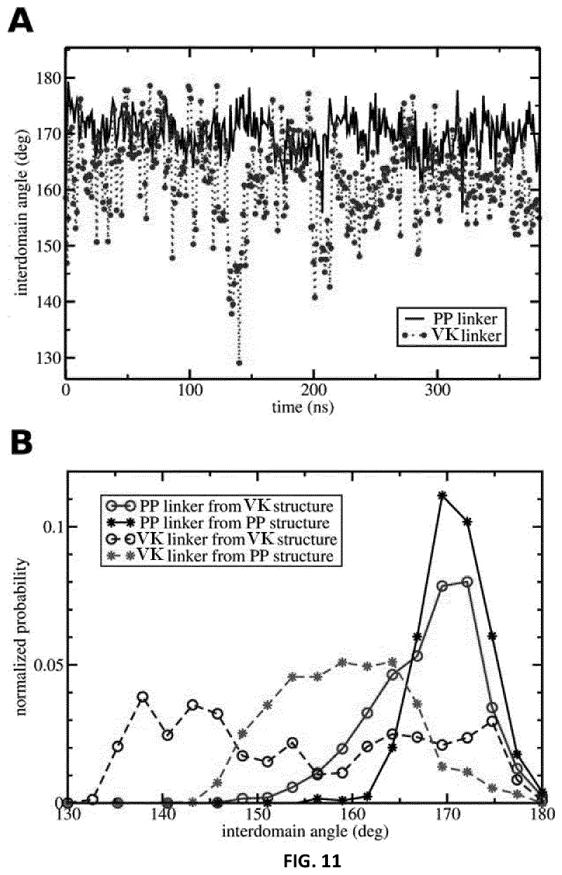

FIG. 11 shows that bending relative to the interdomain angle (angle between the domain—measure of the flexibility of the domains against each other) at the start of the simulation is much larger for the original macrobody construct with a VK linker (up to 40 degrees) whereas for the new linker made of prolines only little bending occurs.

In FIG. 11A, a time-series of the simulations show that the interdomain angle for the VK-linker oscillates with much larger amplitudes than the PP-linker. In FIG. 11B, a histogram is shown of the four simulations with combinations of linker and structure, showing that the interdomain angles in the PP-linker simulations converge toward 167.6 and 169.5 deg when initiated from the VK, and PP structures, respectively. The results indicate that the initial coordinates did not impact significantly the equilibrium interdomain angle, which is found to converge to the same value. This angle is in good agreement with the angle observed in the X-ray structure of the PP-linker fusion polypeptide with 172.6 deg. The standard deviation in these simulations for the interdomain angle was 5.6° for the VK→PP linker simulation, and 3.6° for the simulation starting from the PP structure. As expected, the low standard deviation indicates a much more rigid structure with the PP-linker compared to the simulations done with the VK-linker. Simulations with a VK-linker, starting from either PP or VK structures, are shown to not converge to a stable angle distribution in the time of the simulations, but display a much larger range of values.

Evaluation of Results

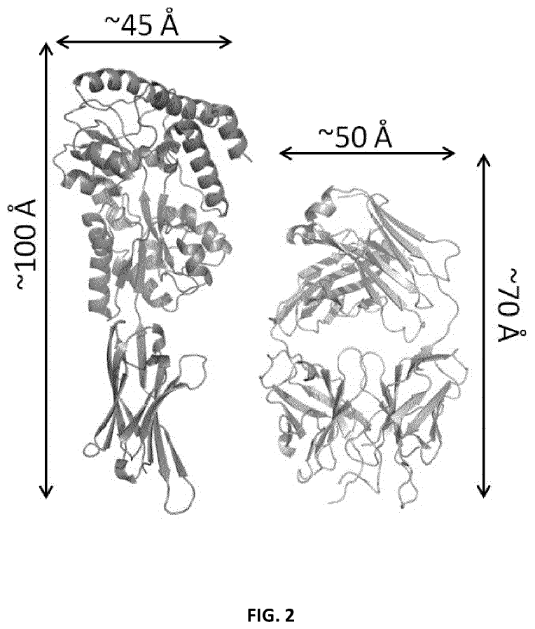

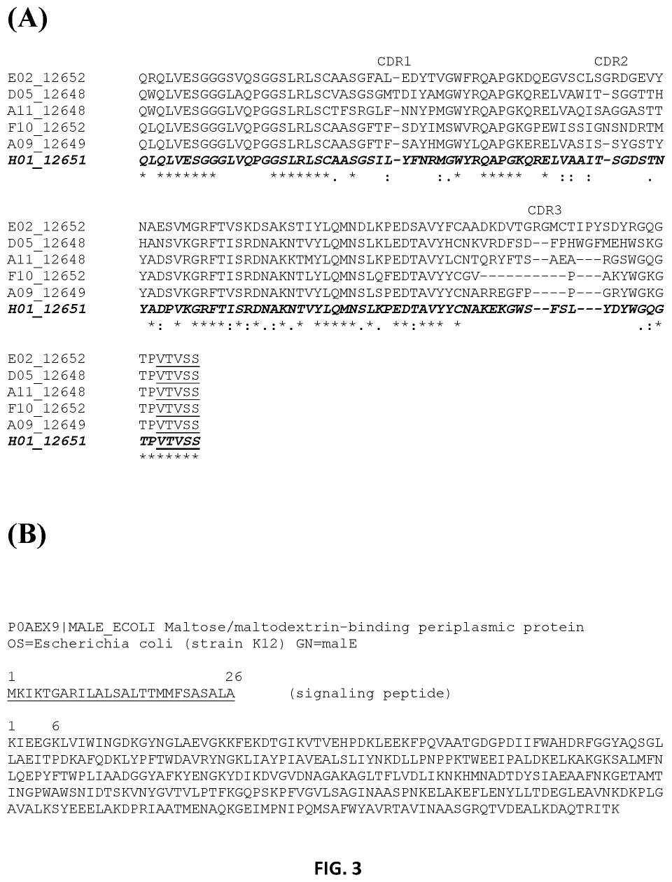

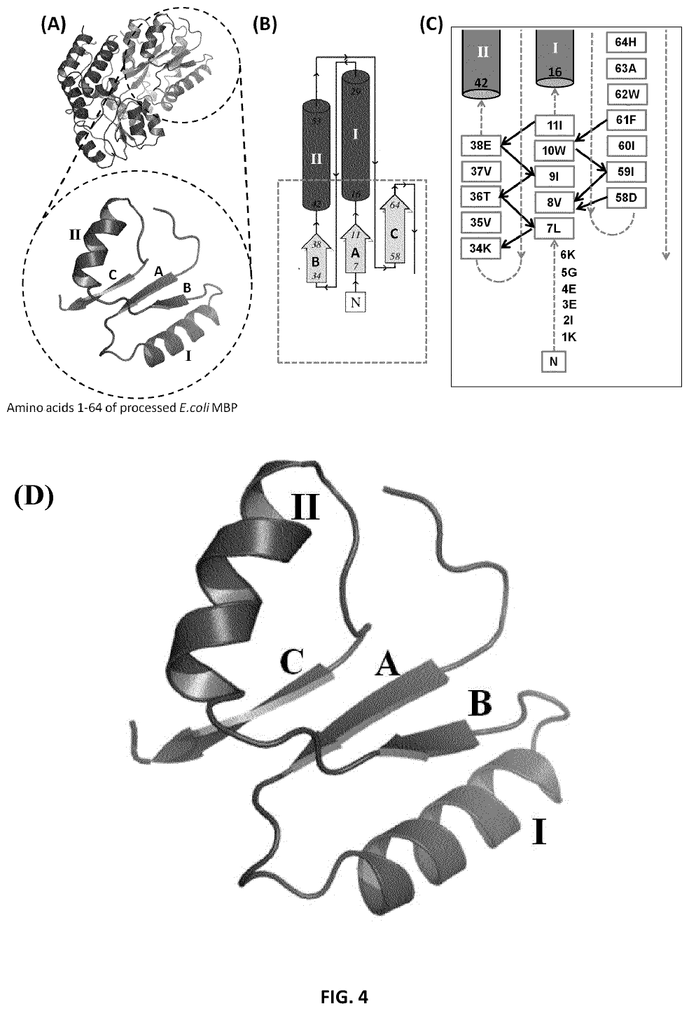

The analysis revealed that the two fused polypeptides with a VK linker have a large conformational freedom and can rotate around the polypeptide backbone at the valine-lysine linker, even at timescales of 500 ns. Both, Val121 and Lysine 6 of MBP (Lys122 in the chimeric fusion polypeptide) were not or at least insufficiently stabilized by hydrogen bond interactions within beta-sheets to prevent rotations. In solution, other than in a protein crystal, the chimeric fusion polypeptide from the structure 6HD8 (FIG. 2) would tumble and on average be present in a number of different conformational states (FIGS. 9-11). If the VHH antigen binding domain would be attached to its respective antigen, the MBP moiety would not be rigidly connected but instead swing around with rather large flexibility. The chimeric fusion polypeptide would thus not fulfill the requirements to serve as a molecular chaperone for protein complexes in solution, e.g. in single particle cryo-microscopy. To increase the rigidity between the VHH antigen binding domain and the MBP moiety, a linker made of prolines was tested (FIG. 8).

Proline is restricted in its rotation due to its unique linkage of the side chain to the backbone. It is an amino acid with exceptional conformational rigidity. Due to the linkage of the α-amino group directly to the main chain, the α-carbon becomes a direct substituent of the side-chain. L-Proline has therefore no freedom of rotation like all other natural amino acids. L-Proline would, from the standpoint of its rigidity, be a suitable amino acid to be tested. However, L-Proline in polypeptides is introducing kinks and its nitrogen-atom cannot contribute as a donor in hydrogen-bonds, only as an acceptor. This is leading to destabilization or termination of alpha-helical or beta-sheet secondary structures. The impact of L-Proline residue to the stability of the linkage between a VHH antigen-binding domain and the MBP domain was unclear, especially due to its influence on secondary structure elements that are essential in the said fusion-polypeptide but also due to the introduced kink(s) that would impact the desired elongated shape of the fusion-polypeptide.

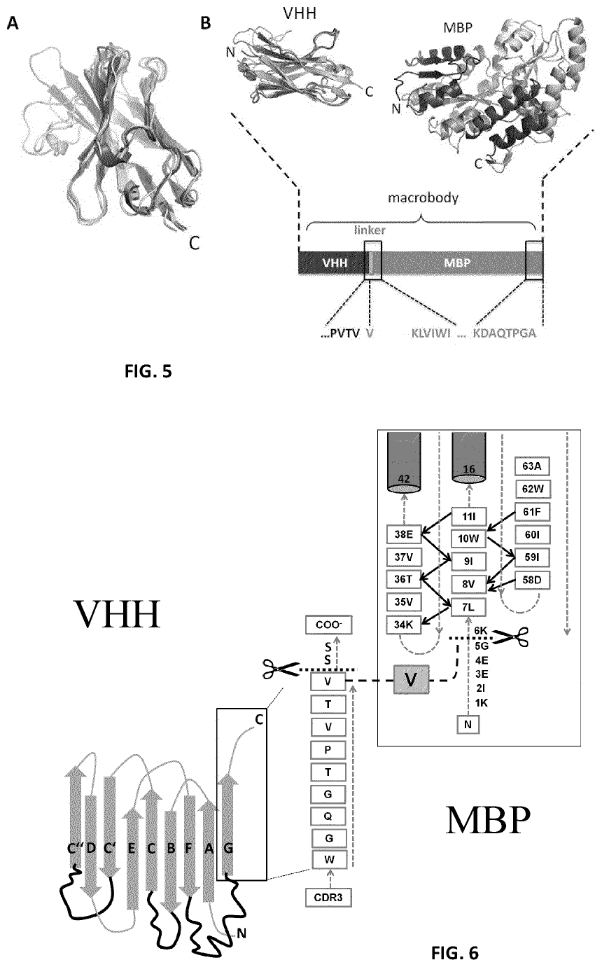

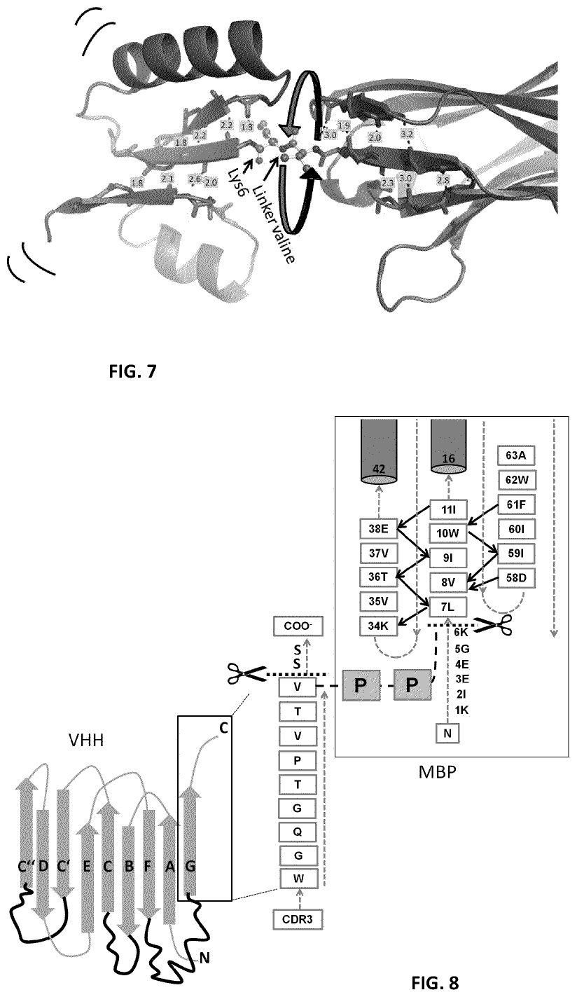

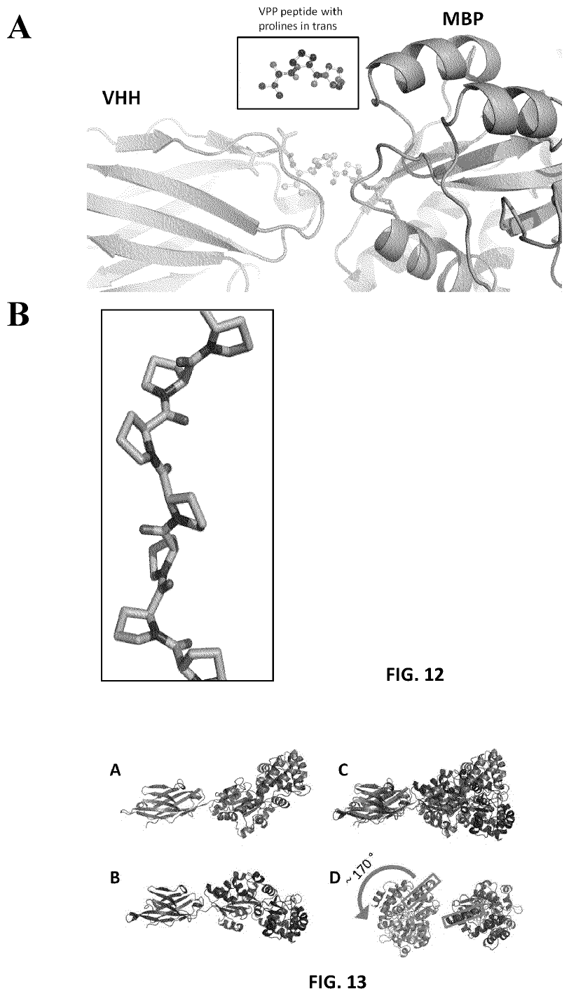

When the linker sequence was changed from . . . VTVVKLI . . . as shown in FIG. 6 to . . . VTVPPLI . . . (FIG. 8) the protein adopted a new conformation in molecular dynamics simulations (italics: C-terminal residues from the VHH domain; bold and underlined (newly introduced linker amino acids; standard letters: N-terminal residues from MBP). The two proline residues show a trans-configuration as in most proteins where two subsequent prolines appear (FIG. 12A) and resembles a short stretch of a polyproline II helix (FIG. 12B). The turns that are introduced at the linker by two consecutive proline-residues lead to a rotation of the MBP-moiety by ˜170 degrees along its major axis (counter-clockwise when viewed from the end opposing the VHH antigen binding domain) (FIG. 13B) in comparison to the original crystal structure in 6HD8 (FIGS. 13A,D). The shape of the protein remained elongated, an optimal configuration because the enlarging moiety of the MBP is on the opposite side of the paratope and thus as far as possible from the epitope. FIG. 13C shows a superposition of FIGS. 13A and B respectively.

To our surprise, the modification with a di-proline linker resulted in a major conformational and structural change compared to the structure of the fusion polypeptide in the structure 6HD8. Most remarkably, this new protein is very stable with respect to relative motions between the VHH antigen binding domain and the MBP. This is apparent when comparing the trajectories in molecular dynamics simulations of the original chimeric fusion polypeptide with a VK linker and from the new fusion polypeptide with a di-Proline (PP) linker (FIGS. 9-11). The bending of the two constructs is compared in FIG. 11 and shows that the di-Proline linker reduces bending between the two fused moieties substantially. Only bending of ±3.6° (within a 95% confidence interval) can be observed, whereas in 6HD8 bending motions of up to 40 degrees can be observed. Furthermore torsional motions are also greatly reduced as seen in FIG. 10 (normal mode analysis). In summary, only minor vibrational motions can be observed. In the next section the X-ray structure of a chimeric fusion polypeptide made of a VHH antigen binding domain and an MBP moiety connected with a di-proline linker is described that confirms the findings of this section.

### EXAMPLE II

### Structure Determination of a Chimeric Fusion Polypeptide with a Di-Proline Linker Between the Fused Moieties by X-Ray Crystallography

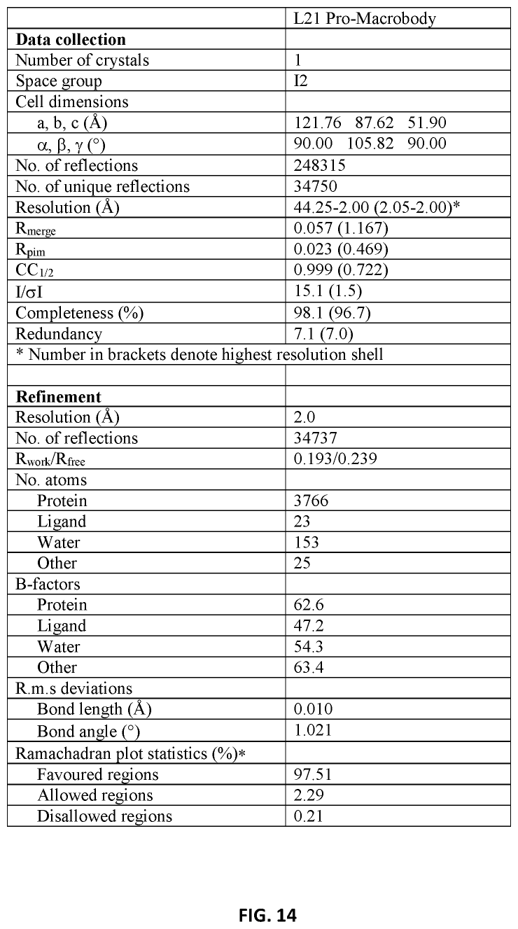

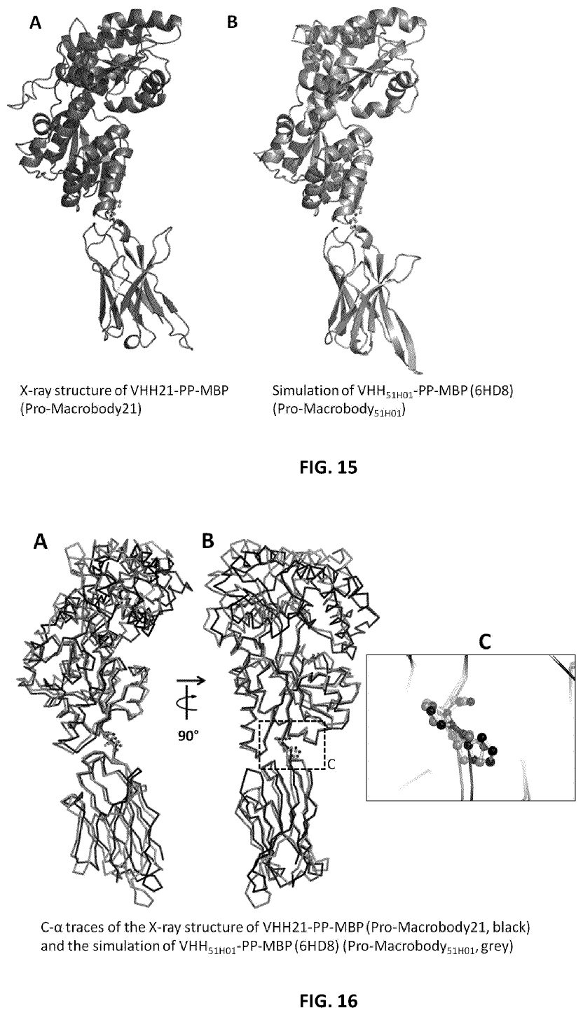

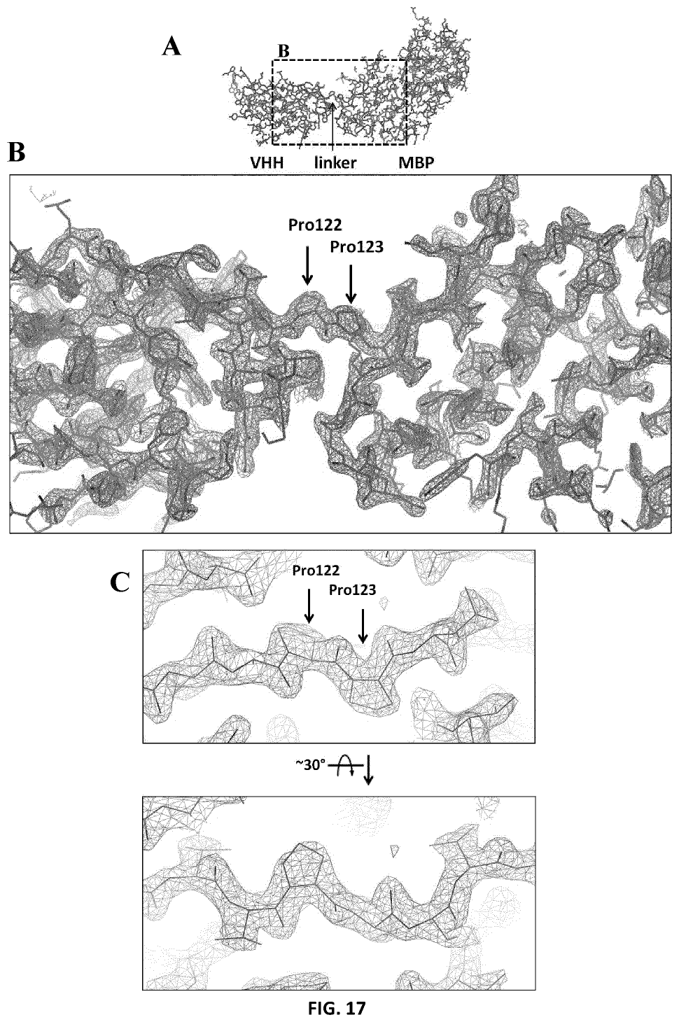

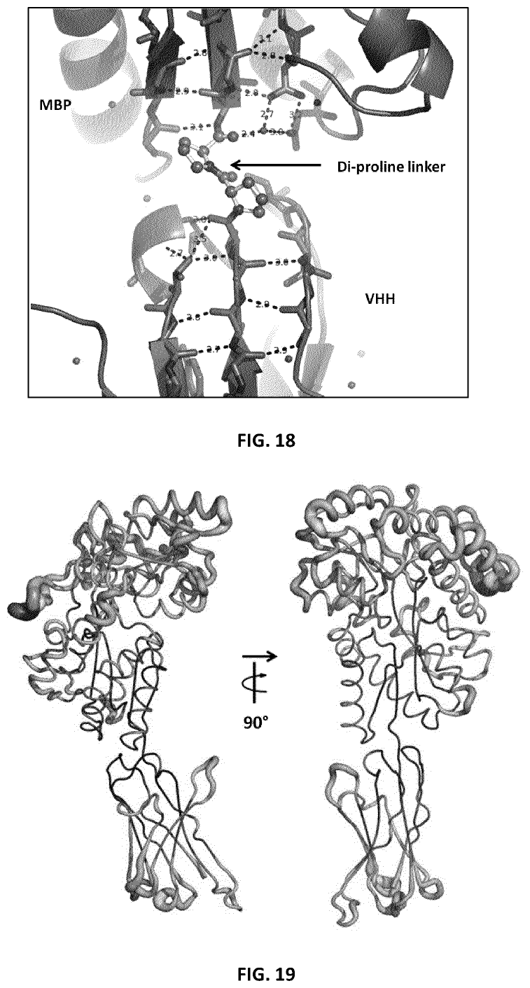

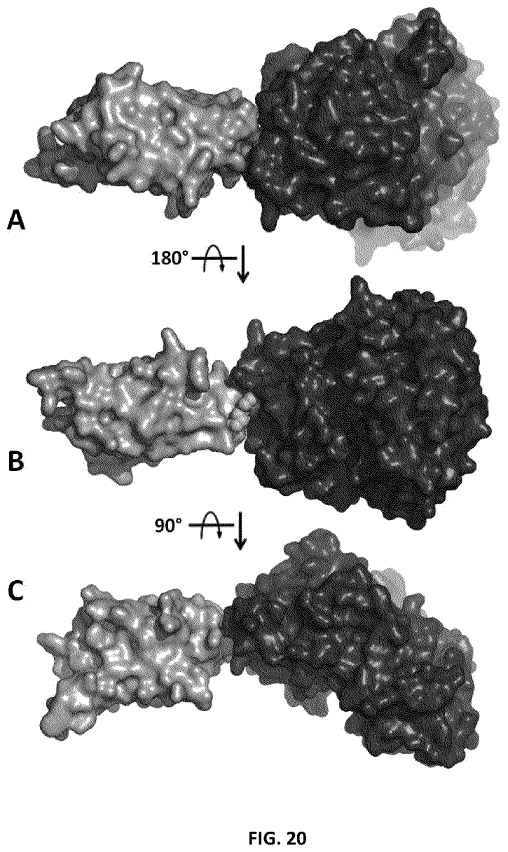

Two confirm the predicted structure, the new fusion polypeptide (FIG. 8) (where the clone of the VHH antigen binding domain is L21, SEQ ID No. 1) was recombinantly produced in Escherichia coli and purified to homogeneity by chromatography (Brunner J. D. et al., BioRxiv 480863, 2018, https://doi.org/10.1101/480863) in the same way as the original fusion polypeptide with a valine linker. The purified protein with the PP linker was concentrated to 10 mg/ml and subjected to crystallization. Crystals were grown, cryo-protected, flash-frozen in liquid nitrogen and subjected to X-ray diffraction at the Swiss Light Source of the Paul Scherrer Institute (beamline X10SA). Crystals diffracted to a resolution of 2.0 Å and belong to the space group I2. The structure was solved by molecular replacement and refined with excellent statistics (FIG. 14). All residues could be unambiguously assigned in the electron density map. The structure is remarkably similar to the predicted structure from the computer simulations as described (FIGS. 15 and 16). The high definition of the electron density map around the linker region is depicted in FIG. 17. A closer view on the linker region reveals the preservation of hydrogen-bonds in the C-terminal end of the VHH domain and the N-terminal domain of the fused MBP (FIG. 18). In the MBP part, the first amino acid is a leucine (leucine 7) and stabilized in a beta-strand. In the VHH domain, the last amino acid that precedes the di-Proline linker is a valine that also interacts with hydrogen-bonds of the main chain in a beta-strand. The di-proline-motif thus serves as a rigid bridge between two rigid parts and the result is a rigid fusion polypeptide overall. This is further reflected by the very low relative B-factors (temperature factors) of the linker region in the crystal structure (FIG. 19). The distribution of the B-factors reveals that motions in the protein are lowest around the linker region and highest at the C-terminus of MBP. This distribution is strikingly similar to the results from the normal mode analysis seen in FIG. 10. In addition, very little space remains between the VHH antigen binding domain and the MBP. The two surfaces of the proteins fit very well two each other (FIG. 20), with no observable clashes but also without superfluous space such that shortening of the linker would be possible or provide a further advantage. The polyproline helix (PPII helix) can be found in protein structures and engineered PPII-helices have been used as molecular rulers to study protein interactions and interdomain FRET signals [Bonger et al. 2010; Adzhubei et al., 2013; Dobitz et al. 2017]. However, there is no report of a fusion polypeptide with a di-proline linker that serves the function of a molecular chaperone for structural biology, or with a related function, and that was created with the purpose of a rigid connection between the fusion partners.

### EXAMPLE III

### The Chimeric Fusion Polypeptide with a Di-Proline Linker Retains the Antigen Binding Properties of the Coupled VHH Antigen Binding Domain

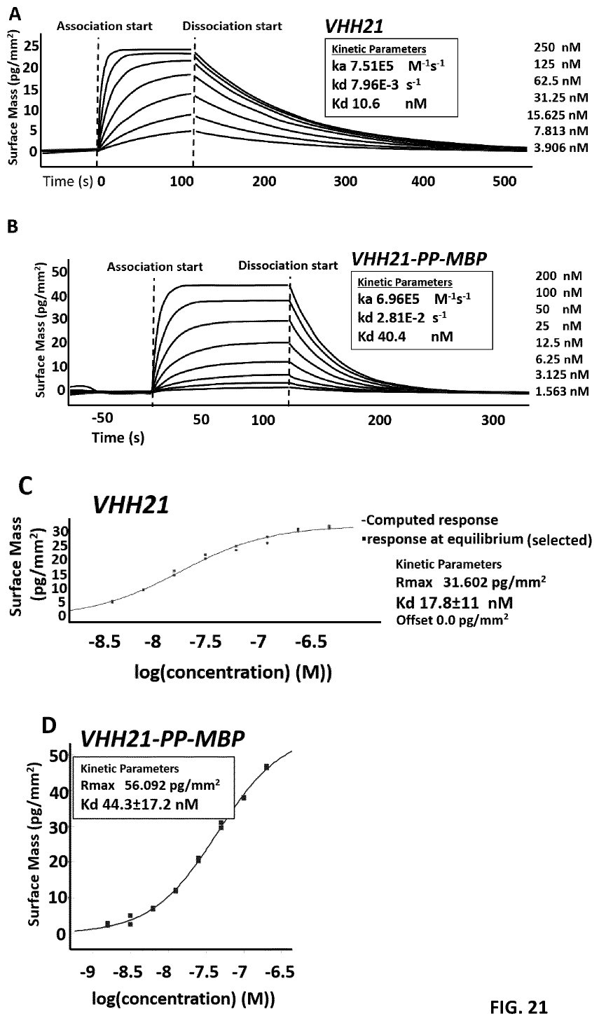

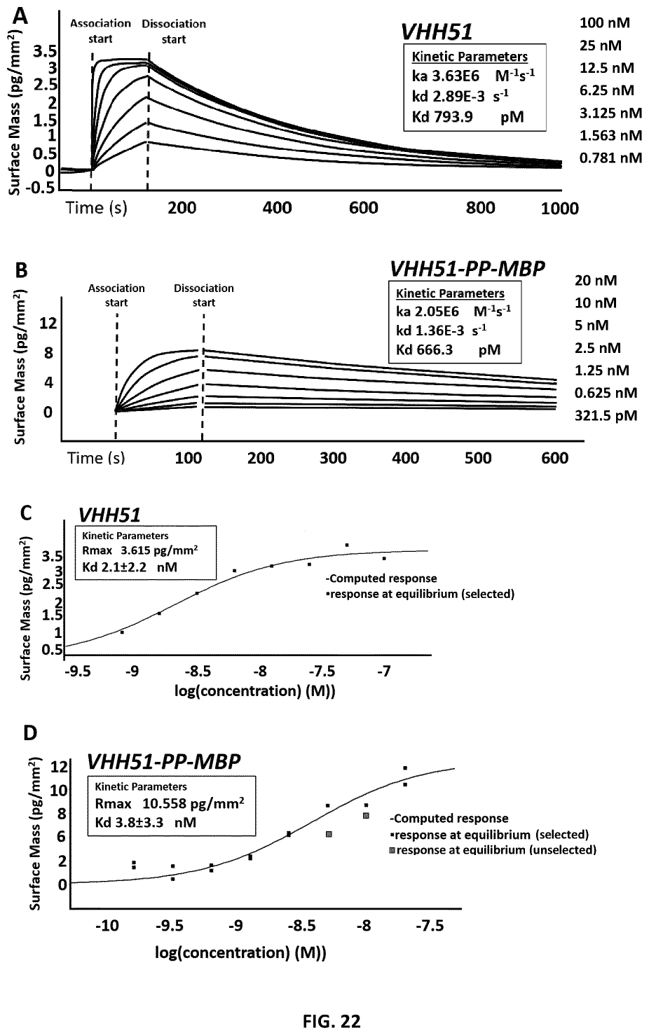

Binding of the VHH antigen binding domain to the respective antigen was measured with waveguide interferometry, a biophysical method to investigate the direct ligand binding to an immobilized molecular target protein enabling evaluation of affinity, stoichiometry and kinetics of binding. The binding kinetics of the unfused VHH antigen binding domains was measured towards the antigen which was coupled to the sensor chip by a biotin-neutravidin interaction according to manufacturer's instructions (Creoptix, Wädenswil, Switzerland). The binding kinetics of the corresponding VHH antigen binding domain in a chimeric fusion protein with MBP and connected by a di-proline linker were also measured and compared (FIGS. 21 and 22). All kinetic parameters (ka, kd and KD) are very similar also for the equilibrium kinetics. In FIG. 21 VHH21-PP-MBP is compared to the VHH21 and in FIG. 22 VHH51-PP-MBP is compared to the VHH51. Both VHH antigen binding domains are directed against NgLptDE and were used for cryo-EM as described below. The data shows that the chimeric fusion of the VHH antigen binding domain with the MBP does not alter the binding to the respective antigen, an essential parameter when transforming a VHH antigen binding domain to a di-proline linked chimeric fusion polypeptide.

### EXAMPLE FOR THE APPLICATION OF PRO-MACROBODIES

### EXAMPLE IV

### A Complex of a Small Membrane Protein and the Chimeric Fusion Polypeptide With a Di-Proline Linker was Purified and Analyzed By Single Particle Cryo-EM

A complex of a chimeric fusion polypeptide with the di proline linker and the corresponding antigen (a bacterial transporter for lipopolysaccharides (LPS)) was prepared. The bacterial LPS transporter-complex LptDE of Neisseria gonnorhoeae (consisting of the proteins LptD (Uniprot) and LptE (Uniprot) was selected as example (NgLptDE). The protein is a relatively small membrane protein target for cryo-EM with only 110 kDa, asymmetric and built almost exclusively of β-strands. These properties make NgLptDE a very suitable example for examining positive impact of Pro-macrobodies regarding structure determination. A low resolution structure that was acquired beforehand without Pro-Macrobodies served as a control for direct comparison. VHH antigen binding domains binding NgLptDE were raised and already characterized beforehand. Two specific VHH antigen binding domains were chosen and transformed to Pro-macrobodies. These are clones L21 and clone L51.

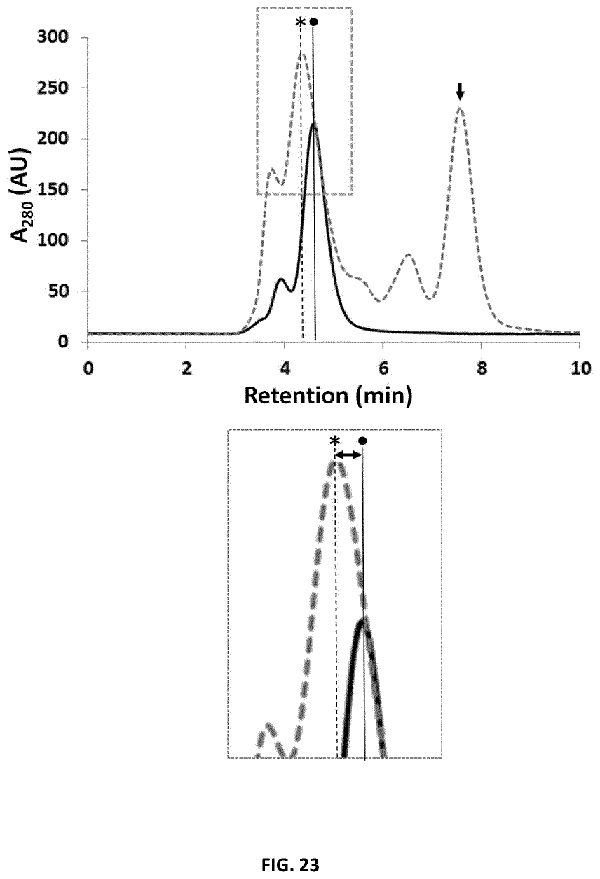

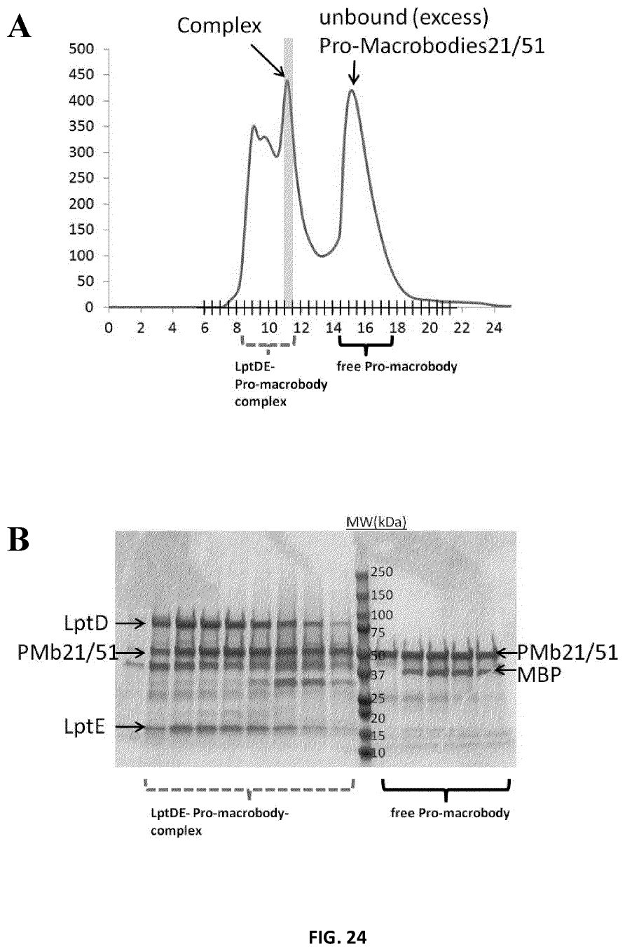

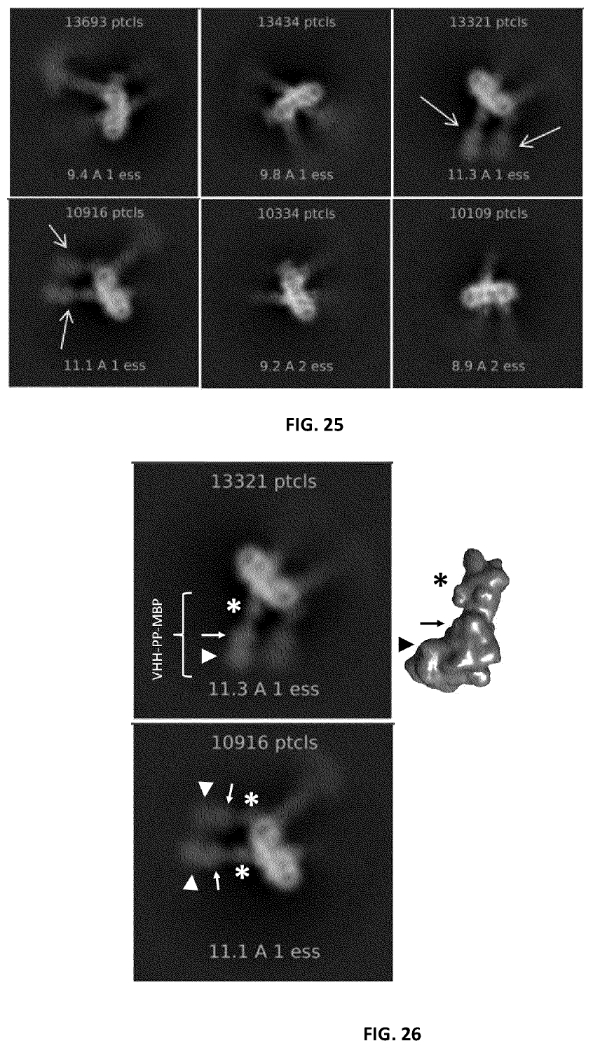

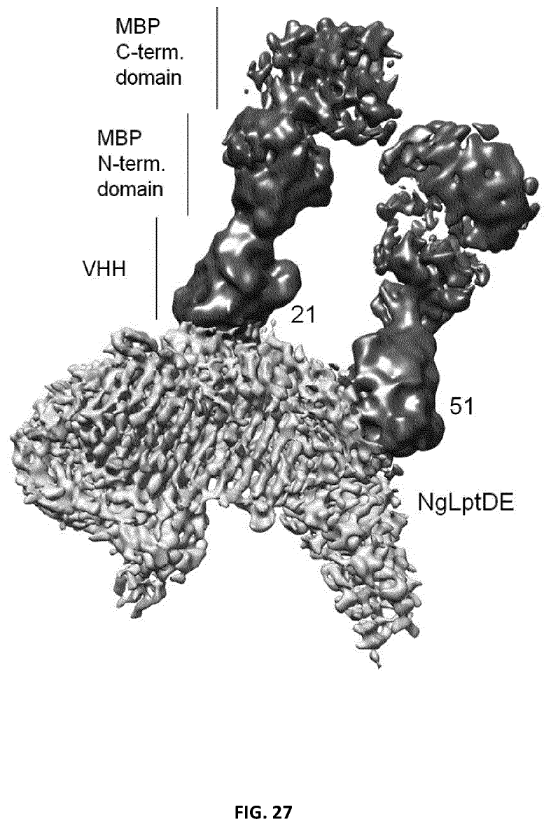

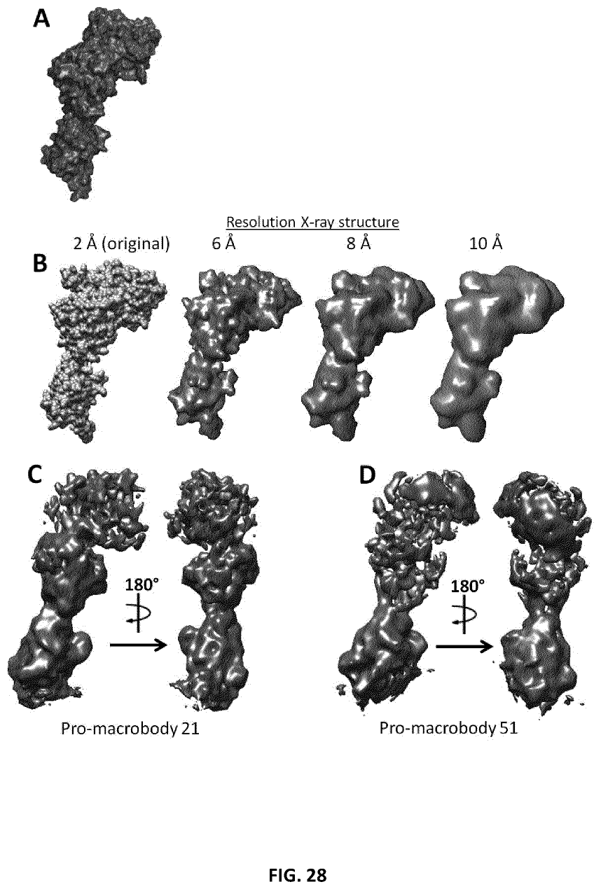

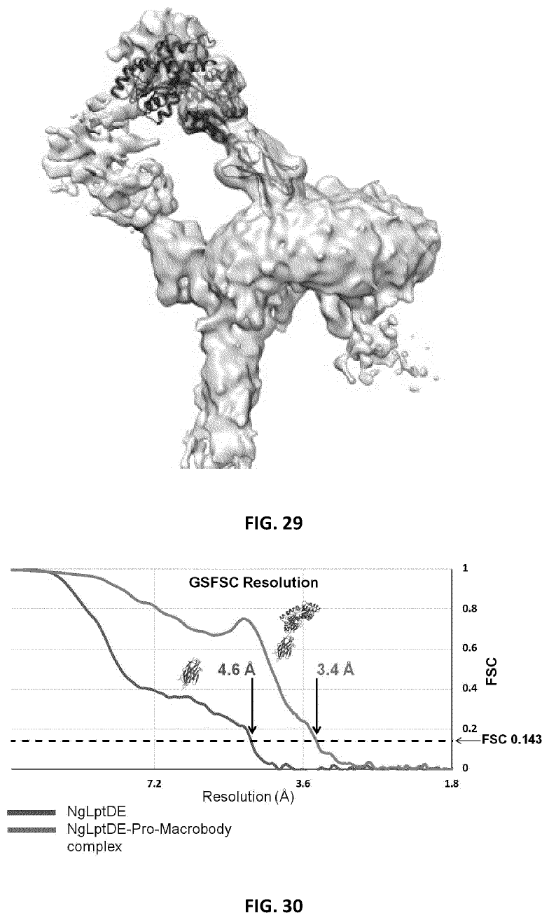

The complex of NgLptDE and the Pro-Macrobodies was purified—after mixing the two components—by size-exclusion chromatography (FIG. 23) and fractionation of the eluate. Several complexes were prepared with one of the two Pro-Macrobodies or both (ternary complex) for cryo-EM. The peak fractions containing the complex of the transport protein and the chimeric fusion polypeptide were analysed by SDS-PAGE (sodium dodecyl sulfate polyacrylamide gel electrophoresis) (FIG. 24). The fraction with the complex protein at a concentration of 1 mg/ml was applied to cryo-EM grids for vitrification in liquid ethane (ternary complex). The grids were examined in a screening electron microscope for quality and then subjected for imaging of micrographs at a Titan Krios electron cryo-microscope at C-CINA/Basel, Switzerland (FEI/ThermoFisher), a 300 kV class microscope that is operated at cryogenic temperature and equipped with a K2 direct electron detector camera (Gatan/ThermoFisher,USA). Particles from the micrograph images were picked in automated manner using CryoSparc software and classified into subclasses (different views on the three-dimensional particles depending on their orientation in the vitrified ice) from which averages (called 2D-classes, as these are projections of a three-dimensional object) are generated automatically by summing up individual particles. From 2D-classes the three-dimensional volume of the object (here a protein complex) can be calculated. In 2D-classes (subclass averages) already some details of the object appear, depending on the orientation (FIG. 25) and provide information about the sample quality and also flexibility of the object. In case of a flexible sub-domain in the object details are washed-out in the sum-average because the flexible part was frozen in many different conformations. If a subdomain is rigidly connected to the object, it will be clearly discernible. Importantly, a rigidly connected subdomain of an object is also providing information on the relative orientation of the object in space, and increases the contrast of the object in electron micrographs. Thereby the classification process is more accurate and particles will be correctly assigned to subclasses which will increase the quality of the calculated volume of the object. The invention is precisely aiming at the provision of such an additional sub-domain that is a rigidly connected particle to an otherwise featureless or too small object for correct classification. In subclass averages of the complex (consisting of the outer membrane NgLptDE transporter and both of the chimeric fusion polypeptides with di-Proline linker) the chimeric fusion polypeptides are clearly visible. It is further a relatively well defined structure, where the two domains (N-terminal and C-terminal domain) of the MBP-moieties are already discernible (FIG. 26). This is in strong support of a rigid molecule, since it would otherwise be “washed out” in the images due to motions. When a three-dimensional volume was reconstructed using the 2D-class averages it was possible to generate a map that shows resolved chimeric fusion polypeptides attached to the target protein (FIG. 27). The structure of the chimeric fusion polypeptide with di-proline linker is almost identical to the structure solved by X-ray crystallography and also to the structure prediction from the molecular dynamics simulation (FIG. 28). This consistently proofs that the newly designed chimeric fusion polypeptide of a VHH domain connected to the maltose binding protein malE of E. coli by a peptide linker of two prolines is indeed folded as predicted and is a rigid entity in solution. The chimeric fusion polypeptide models from the X-ray structure (FIG. 15A) can easily be fitted into the density of the map derived from electron microscopy as depicted in FIG. 29. Furthermore, both, Pro-Macrobodies L21 and L51, albeit binding to different epitopes share the same fold and are equally well resolved in the three-dimensional reconstruction. This illustrates that the Pro-Macrobody-format can be applied to different VHH antigen binding domains and has a universal character.

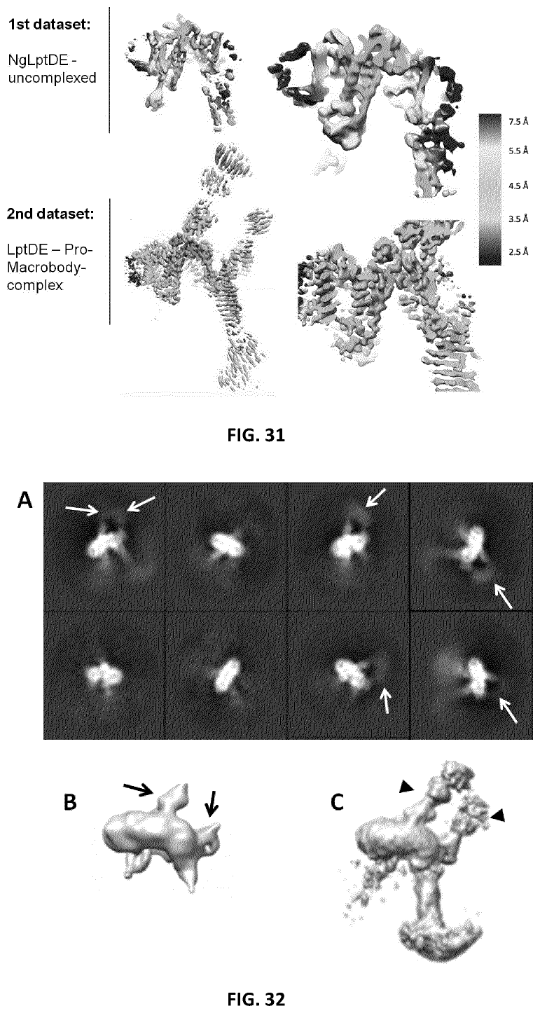

The invention enables higher resolution EM maps for target proteins. A difference of 1.2 Angstrom could be observed for the resolution of density maps of the bacterial transporter in the absence or presence of the specific chimeric fusion polypeptide (i.e. uncomplexed compared to complexed transporter with two Pro-Macrobodies). Applying the gold standard fourier shell correlation (GSFSC), the standard measure to obtain the global resolution of a given density map from electron microscopic volume reconstructions (at a FSC cutoff of 0.143) is 4.6 Å for the uncomplexed sample and 3.4 Å for the complex of the transporter with the chimeric fusion polypeptide (FIG. 30). In FIG. 31 the improved resolution of the sample complexed with the Pro-Macrobody is compared with the sample without chimeric fusion polypeptide. This data provides a proof of concept for the usefulness and effective increase in resolution of target proteins by applying the chimeric fusion polypeptide of an antigen binding domain connected with a di-proline linker to MBP, which is termed hereafter “Pro-Macrobody”.

The fusion protein with the di-proline linker as described in examples I-IV is thus a unique design because one domain (the VHH domain) can be exchanged without sacrificing the rigidity of the fusion protein. It can be assumed that a single polypeptide linker is always flexible, unless the two connected domains show specific interactions at the interface, such that attempts to stabilize would fail. Using a fusion protein with the di-proline linker we generated rigidly connected two non-interacting proteins and the double proline linker identified ideally serves the purpose of (i) a rigid connection, and (ii) keeping the VHH domain freely exchangeable.

It is important to note that the rigidity of the chaperone becomes increasingly relevant with a decrease of the protein target's size demanding a highly rigid chaperone. The chimeric fusion polypeptide with a di-proline linker described here represents a significant technical improvement of molecular chaperones for structural biology.

The full amino-acid sequence of the chimeric fusion polypeptide of a VHH antigen binding domain (clone L21) and MBP with the di proline linker (Pro-Macrobody 21) is SEQ ID 001:

GPSQVQLVESGGGLVQAGGSLRLSCAASGFPVKYEHMYWYRQAPGKEREWV
AAINSAGNETHYADSVKGRFTISRDNAKNTVYLQMNSLKPEDTAVYYCNVK
DIGWWAAYDYWGQGTQVTVPPLVIWINGDKGYNGLAEVGKKFEKDTGIKVT
VEHPDKLEEKFPQVAATGDGPDIIFWAHDRFGGYAQSGLLAEITPDKAFQD
KLYPFTWDAVRYNGKLIAYPIAVEALSLIYNKDLLPNPPKTWEEIPALDKE
LKAKGKSALMFNLQEPYFTWPLIAADGGYAFKYENGKYDIKDVGVDNAGAK
AGLTFLVDLIKNKHMNADTDYSIAEAAFNKGETAMTINGPWAWSNIDTSKV
NYGVTVLPTFKGQPSKPFVGVLSAGINAASPNKELAKEFLENYLLTDEGLE
AVNKDKPLGAVALKSYEEELAKDPRIAATMENAQKGEIMPNIPQMSAFWYA
VRTAVINAASGRQTVDEALKDAQTPG

The full amino-acid sequence of the chimeric fusion polypeptide of a VHH antigen binding domain (clone L51) and MBP with the di proline linker (Pro-Macrobody 51) is SEQ ID 002:

GPSQVQLVESGGGSVQAGGSLRLSCAASGSISSITYLGWFRQAPGKEREGV
AALATYYGHTYYADSVKGRFTVSLDNAKNTVYLQMNSLKPEDTALYYCAAA
YSGIWTPLGVWATYEYWGQGTQVTVPPLVIWINGDKGYNGLAEVGKKFEKD
TGIKVTVEHPDKLEEKFPQVAATGDGPDIIFWAHDRFGGYAQSGLLAEITP
DKAFQDKLYPFTWDAVRYNGKLIAYPIAVEALSLIYNKDLLPNPPKTWEEI
PALDKELKAKGKSALMFNLQEPYFTWPLIAADGGYAFKYENGKYDIKDVGV
DNAGAKAGLTFLVDLIKNKHMNADTDYSIAEAAFNKGETAMTINGPWAWSN
IDTSKVNYGVTVLPTFKGQPSKPFVGVLSAGINAASPNKELAKEFLENYLL
TDEGLEAVNKDKPLGAVALKSYEEELAKDPRIAATMENAQKGEIMPNIPQM
SAFWYAVRTAVINAASGRQTVDEALKDAQTPG

The VHH domain (antigen-binding) is shown in italics and can be exchanged for any other VHH because of the conserved structure. The linker is shown in bold and underlined. The remaining sequence is the enlarging domain (malE from E. coli). The VHH domain shown here is the antigen-binding domain clone 21 that was crystallized and used for electron microscopy.

In a final experiment the Pro-Macrobodies were compared with macrobodies (comprising a VK-linker as in the PDB entry 6HD8). For this NgLptDE was complexed with macrobodies 21 and 51 (identical VHH domains as in the respective Pro-Macrobodies), purified by chromatography in the same manner and subjected to analysis by cryo-EM. It became apparent that macrobodies could not improve the resolution significantly in comparison to uncomplexed NgLptDE as shown in FIG. 32. The MBP moiety, if not connected with a PP linker is not well resolved due to high intrinsic motions at the linker. In three-dimensional reconstructions from this data-set no clear density for the MBP moiety was apparent. The overall resolution was consequently not much improved. This experiment shows that the linkage using two prolines as in Pro-Macrobodies is resulting in a significant advantage over previous macrobodies.

1. A fusion polypeptide comprising a first polypeptide which is an antigen-binding domain and a second polypeptide which is a polypeptide-scaffold, wherein the polypeptide-scaffold comprises parallel or antiparallel beta-strands and the antigen binding domain is linked by a peptide linker at its C-terminus to the N-terminus of the polypeptide-scaffold and wherein said peptide linker consists of one or more proline residues.

2. A fusion polypeptide according to claim 1, wherein the polypeptide-scaffold is a maltose binding protein.

3. A fusion polypeptide according to claim 1 or 2 wherein the polypeptide-scaffold is Escherichia coli maltose binding protein, Uniprot entry P0AEX9.

4. A fusion polypeptide according to any preceding claim, wherein the peptide linker consists of one, two, three or four proline residues.

5. A fusion polypeptide according to any preceding claim, wherein the polypeptide-scaffold comprises a polypeptide of the superfamily of periplasmic binding proteins of Interpro entry IPR025997, or
a polypeptide of the superfamily of periplasmic binding protein-like I integral of Interpro entry IPR028082, or
a periplasmic binding protein domain Pfam domain Peripla_BP_4of PF13407.

6. A fusion polypeptide according to any preceding claim, wherein the antigen binding domain comprises an immunoglobulin-like fold.

7. A fusion polypeptide according to any preceding claim, wherein the antigen-binding domain comprises an immunoglobulin (Ig) domain.

8. A fusion polypeptide according to any preceding claim, wherein the antigen-binding domain is a VHH antigen-binding domain.

9. A fusion polypeptide according to any preceding claim, wherein the antigen-binding domain comprises a camelid VHH or elasmobranch VHH, or shark VHH, or ray VHH or skate VHH or sawfish VHH or VHH domains from heavy or light chain of mammalian antibodies or monobodies.

10. An amino acid sequence encoding the fusion polypeptide of anyone of claims 1-10.

11. An amino acid sequence according to claim 10, comprising Sequence ID NO: 001 or Sequence ID NO: 002.

12. A complex comprising:
i) the fusion polypeptide of any one of claims 1-9, and
ii) a target protein, wherein said target protein is specifically bound to said fusion polypeptide.

13. The use of the fusion polypeptide according to anyone of claims 1-9, the amino acid sequence according to claims 10 and 11, and the complex according to claim 12 for structural analyses of a target protein.

14. The use of the fusion polypeptide according to any one of claims 1-9 as a medicine.

15. The use of the fusion polypeptide according to any one of claims 1-9 for diagnostic purposes.
